(19) United States
(12) Patent Application Publication
(10) Pub. No.: US 2026/0309717 A1
(43) Pub. Date: Oct. 8, 2026

(54) PROXIMITY INDUCING SYSTEM AND USES THEREOF

(71) Applicants: SORBONNE UNIVERSITE, PARIS (FR); INSTITUT CURIE, PARIS (FR); CENTRE NATIONAL DE LA RECHERCHE SCIENTIFIQUE (CNRS), PARIS (FR); ECOLE NORMALE SUPERIEURE, PARIS (FR)

(72) Inventors: Arnaud GAUTIER, PARIS (FR); Franck PEREZ, PARIS (FR); Sara BOTTONE, PARIS (FR); Zeyneb Vildan CAKIL, PARIS (FR); Octave JOLIOT, PARIS (FR)

(73) Assignees: SORBONNE UNIVERSITE, PARIS (FR); INSTITUT CURIE, PARIS (FR); CENTRE NATIONAL DE LA RECHERCHE SCIENTIFIQUE (CNRS), PARIS (FR); ECOLE NORMALE SUPERIEURE, PARIS (FR)

(21) Appl. No.: 19/108,208

(22) PCT Filed: Sep. 5, 2023
(86) PCT No.: PCT/EP2023/074340

§ 371 Date: Mar. 3, 2025

(30) Foreign Application Priority Data
Sep. 5, 2022 (EP) 22306308.2

(51) Int. Cl. G01N 33/68; G01N 21/64; G01N 33/58

(52) U.S. Cl. CPC G01N 33/6845; G01N 21/6428; G01N 33/582; G01N 2021/6439

(57) ABSTRACT

A method for inducing proximity between two biological molecules in a sample, to an assay relying on the induction of proximity between two biological molecules in a sample, and to a proximity inducing system including two polypeptides and a molecular inducer of proximity. Also, a cell or cell line including two biological molecules.

### FIELD OF INVENTION

The present invention relates to the control, and optionally the detection, of the localization of biological molecules. In particular, the present invention relates to a proximity inducing system that allows to induce, and optionally detect, proximity between two biological molecules. The present invention also relates to uses of said proximity inducing system.

### BACKGROUND OF INVENTION

Many essential biological processes, such as gene regulation, protein transport, signal transduction, or metabolism, are governed and regulated by the physical closeness/interaction, or proximity, between biological molecules such as proteins and/or nucleic acids. In particular, proximity between proteins plays an essential and ubiquitous role in numerous biological processes.

Molecular tools enabling to control and observe the proximity of proteins are essential for studying the functional role of the physical distance between two proteins. Various molecular tools were thus developed to understand the role of proximity in cellular and physiological mechanisms. Most of these tools allow either to control protein proximity or to observe protein proximity.

In particular, chemically induced proximity (CIP) technologies have been developed to enable the precise temporal control of biological processes such as transcription, cell signaling, or protein localization. The CIP technologies or systems generally rely on the genetic fusion of proteins to dimerization domains that interact in a specific manner in presence of a small molecule, acting as a chemical inducer of proximity. Examples of CIP systems include systems based on the interaction between the FK506 binding protein (FKBP) and the FKBP-rapamycin binding domain (FRB), inducible with rapamycin or synthetic rapamycin analogs of rapamycin called rapalogs.

Of note, most of the CIP systems aim at controlling protein proximity and do not allow the detection of the protein interaction triggered by the chemical inducer of proximity and/or the visualization of the protein localization following proximity induction. Such detection and/or visualization thus usually require the use of an additional reporter, distinct from the interacting dimerization domains and the chemical inducer of proximity. Furthermore, CIP systems usually rely on two dimerization domains of more than 10 kDa each, that might lead to dysfunctional fusion proteins. For example, in rapamycin or rapalogs based CIP, FKBP is about 12 kDa and FRB is about 11 kDa. Finally, most CIP systems rely on chemical inducers of proximity that induce the formation of very tightly bound ternary assemblies. Such ternary assemblies are usually too stable to be dissociated by washing away the inducer, preventing the reversal of the recruitment process.

There is thus a need for improved proximity inducing systems. In particular, there is a need for robust, rapid, and fully reversible proximity inducing systems, allowing for the control, and advantageously the visualization, of protein proximity with high temporal resolution.

The Inventors surprisingly demonstrated that a first polypeptide of 114 amino acid residues having a sequence as set forth in SEQ ID NO: 1 and a second polypeptide of 11 amino acid residues having a sequence as set forth in SEQ ID NO: 2 are able to complement and form a ternary assembly with a small synthetic compound which acts as a molecular inducer of proximity. Indeed, the ternary assembly between the two polypeptide and the small synthetic compound is induced by said small synthetic compound regardless of any initial proximity between the two polypeptides of SEQ ID NO: 1 and SEQ ID NO: 2. Accordingly, in the absence of the molecular inducer of proximity, the two polypeptides of SEQ ID NO: 1 and SEQ ID NO: 2 are not able to complement. The two polypeptides of SEQ ID NO: 1 and SEQ ID NO: 2 and the molecular inducer of proximity thus form a proximity inducing system, that may be used to control the localization of biological molecules such as proteins.

Of note, the molecular inducer of proximity may be a fluorogenic chromophore that emits substantial fluorescence only when interacting with the two polypeptides of SEQ ID NO: 1 and SEQ ID NO: 2. The proximity inducing system may thus advantageously allow detection and/visualization of the ternary assembly without the need for any additional reporter. Furthermore, the Inventors also showed that the ternary assembly between the two polypeptide and the molecular inducer of proximity is extremely rapid and remains stable over a long period of time, unless the molecular inducer of proximity is removed. Indeed, the proximity inducing system is fully reversible and accordingly, can be used to control the proximity between two proteins with a high temporal resolution through addition and removal of the molecular inducer of proximity. As illustrated in the experimental section, through its ability to induce proximity between two biological molecules, and optionally to detect said proximity, the system described herein may be a relevant tool for biological and biomedical applications requiring the control of the localization, transport, stability and/or function of biological molecules.

### SUMMARY

The present invention relates to a method for inducing proximity between two biological molecules in a sample, said method comprising the following steps:
obtaining a first biological molecule coupled to a first polypeptide, wherein said first polypeptide comprises an amino acid sequence as set forth in SEQ ID NO: 1 or an amino acid sequence having at least about 70% identity with SEQ ID NO: 1, or a truncated fragment thereof comprising at least 89 consecutive amino acid residues from the C-terminal end of SEQ ID NO: 1 or of the sequence having at least about 70% identity with SEQ ID NO: 1;
obtaining a second biological molecule coupled to a second polypeptide, wherein said second polypeptide comprises an amino acid sequence as set forth in SEQ ID NO: 2 or an amino acid sequence having at least about 70% identity with SEQ ID NO: 2, or a truncated fragment thereof comprising at least 8 consecutive amino acid residues from the N-terminal end of SEQ ID NO: 2 or of the sequence having at least about 70% identity with SEQ ID NO: 2;
adding a molecular inducer of proximity to the sample;
   wherein, upon addition of the molecular inducer of proximity, the first polypeptide, the second polypeptide and the molecular inducer of proximity form a ternary assembly, thereby inducing proximity between the first and the second biological molecules present in the sample.

The present invention also relates to an assay relying on the induction of proximity between two biological molecules in a sample, said assay comprising the following steps:
obtaining a first biological molecule coupled to a first polypeptide, wherein said first polypeptide comprises an amino acid sequence as set forth in SEQ ID NO: 1 or an amino acid sequence having at least about 70% identity with SEQ ID NO: 1, or a truncated fragment thereof comprising at least 89 consecutive amino acid residues from the C-terminal end of SEQ ID NO: 1 or of the sequence having at least about 70% identity with SEQ ID NO: 1;
obtaining a second biological molecule coupled to a second polypeptide, wherein said second polypeptide comprises an amino acid sequence as set forth in SEQ ID NO: 2 or an amino acid sequence having at least about 70% identity with SEQ ID NO: 2, or a truncated fragment thereof comprising at least 8 consecutive amino acid residues from the N-terminal end of SEQ ID NO: 2 or of the sequence having at least about 70% identity with SEQ ID NO: 2;
adding a molecular inducer of proximity to the sample;
   wherein, upon addition of the molecular inducer of proximity, the first polypeptide, the second polypeptide and the molecular inducer of proximity form a ternary assembly, thereby inducing proximity between the first and the second biological molecules present in the sample.

In some embodiments, the concentration of molecular inducer of proximity required to reach half maximal assembly in the sample is of less than about 5 μM.

In some embodiments, the molecular inducer of proximity is fluorogenic and the method or assay further comprises detecting a fluorescence resulting from the formation of the ternary assembly between the first polypeptide, the second polypeptide and the fluorogenic molecular inducer of proximity.

In some embodiments, the first polypeptide consists of an amino acid sequence as set forth in SEQ ID NO: 1, and the second polypeptide consists of an amino acid sequence as set forth in SEQ ID NO: 2.

In some embodiments, the molecular inducer of proximity is a compound of formula (I)

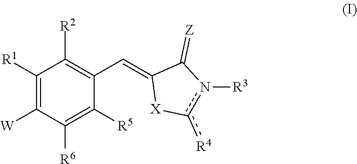

or a salt and/or solvate thereof;
wherein R¹, R², R⁵ and R⁶ each independently represents H, halo, hydroxyl, aryl, alkyl, cycloalkyl, heteroalkyl (e.g., alkoxy) or heterocycloalkyl,
   wherein said aryl, alkyl, cycloalkyl, heteroalkyl or heterocycloalkyl is optionally substituted by at least one group selected from halo, hydroxyl, oxo, nitro, amido, carboxy, amino, cyano, haloalkoxy and haloalkyl;
R³ represents a non-binding doublet (i.e., a free pair of electrons), H, halo, hydroxyl, aryl, alkyl, cycloalkyl, heteroalkyl or heterocycloalkyl,
   wherein said aryl, alkyl, cycloalkyl, heteroalkyl or heterocycloalkyl is optionally substituted by at least one group selected from halo, hydroxyl, oxo, nitro, amido, carboxy, amino, cyano, haloalkoxy and haloalkyl;
R⁴ represents a single or a double bound, interrupted or terminated by one S, O or N heteroatom, wherein said heteroatom is optionally substituted by at least one group selected from H, hydroxyl, aryl, alkyl, cycloalkyl, heteroalkyl and heterocycloalkyl,
   wherein said aryl, alkyl, cycloalkyl, heteroalkyl or heterocycloalkyl is optionally substituted by at least one group selected from halo, hydroxyl, oxo, nitro, amido, carboxy, amino, cyano, haloalkoxy and haloalkyl;
W represents OH, SH, NHR⁷ or NR⁷R⁸, wherein R⁷ and R⁸ each independently represents H, halo, hydroxyl, aryl, alkyl, cycloalkyl, heteroalkyl or heterocycloalkyl,
   wherein said aryl, alkyl, cycloalkyl, heteroalkyl or heterocycloalkyl is optionally substituted by at least one group selected from halo, hydroxyl, oxo, nitro, amido, carboxy, amino, cyano, haloalkoxy and haloalkyl;
X represents O, S or NH; and
Z represents O, S or NH.

In some embodiments, the assay is for controlling the localization, the transport and/or the function of a biological molecule. In some embodiments, the assay is for inducing the degradation of a biological molecule. In some embodiments, the assay is for detecting the co-occurrence of two biological molecules in the sample.

In some embodiments, the first and second biological molecules are proteins.

The present invention also relates to a proximity inducing system comprising two polypeptides and a molecular inducer of proximity, wherein:
a first polypeptide comprises an amino acid sequence as set forth in SEQ ID NO: 1 or an amino acid sequence having at least about 70% identity with SEQ ID NO: 1, or a truncated fragment thereof comprising at least 89 consecutive amino acid residues from the C-terminal end of SEQ ID NO: 1 or of the sequence having at least about 70% identity with SEQ ID NO: 1; and
a second polypeptide comprises an amino acid sequence as set forth in SEQ ID NO: 2 or an amino acid sequence having at least about 70% identity with SEQ ID NO: 2, or a truncated fragment thereof comprising at least 8 consecutive amino acid residues from the N-terminal end of SEQ ID NO: 2 or of the sequence having at least about 70% identity with SEQ ID NO: 2,
   wherein the first polypeptide, the second polypeptide and the molecular inducer of proximity form a ternary assembly.

In some embodiments, the concentration of molecular inducer of proximity required to reach half maximal assembly is of less than about 5 μM. In some embodiments, the molecular inducer of proximity is fluorogenic. In some embodiments, the molecular inducer of proximity is a compound of formula (I) as described above, or a salt and/or solvate thereof.

In some embodiments, the first polypeptide consists of an amino acid sequence as set forth in SEQ ID NO: 1, and the second polypeptide consists of an amino acid sequence as set forth in SEQ ID NO: 2.

The present invention also relates to a cell or cell line comprising two biological molecules, wherein:
a first biological molecule is coupled to a first polypeptide, wherein said first polypeptide comprises an amino acid sequence as set forth in SEQ ID NO: 1 or an amino acid sequence having at least about 70% identity with SEQ ID NO: 1, or a truncated fragment thereof comprising at least 89 consecutive amino acid residues from the C-terminal end of SEQ ID NO: 1 or of the sequence having at least about 70% identity with SEQ ID NO: 1;
a second biological molecule is coupled to a second polypeptide, wherein said second polypeptide comprises an amino acid sequence as set forth in SEQ ID NO: 2 or an amino acid sequence having at least about 70% identity with SEQ ID NO: 2, or a truncated fragment thereof comprising at least 8 consecutive amino acid residues from the N-terminal end of SEQ ID NO: 2 or of the sequence having at least about 70% identity with SEQ ID NO: 2,
   wherein in presence of a molecular inducer of proximity, the first polypeptide, the second polypeptide and the molecular inducer of proximity are able to form a ternary assembly.

In some embodiments, the concentration of molecular inducer of proximity required to reach half maximal assembly is of less than about 5 μM. In some embodiments, the first and second biological molecule are proteins.

### Definitions

In the present invention, the following terms have the following meanings:

### Chemical Definitions

“Alkyl”, by itself or as part of another group, refers to a hydrocarbyl radical of formula CₙH₂ₙ₊₁ wherein n is a number greater than or equal to 1. Generally, alkyl groups of this invention comprise from 1 to 12 carbon atoms, preferably from 1 to 6 carbon atoms. Alkyl groups may be linear or branched and may be substituted as indicated herein. Non-limiting examples of alkyl groups include methyl, ethyl, propyl (n-propyl, i-propyl), butyl (n-butyl, i-butyl, s-butyl and t-butyl), pentyl and its isomers (e.g., n-pentyl, iso-pentyl), and hexyl and its isomers (e.g., n-hexyl, iso-hexyl).

“Alkoxy” refers to any —O-alkyl or —O-aryl group. Generally, alkoxy groups of this invention are —O-alkyl groups.

“Amido” refers to —CONH₂ group.

“Amino” refers to —NH₂ group.

“Aryl” refers to a polyunsaturated, aromatic hydrocarbyl group having a single ring (i.e., phenyl) or multiple aromatic rings fused together (e.g., naphtyl) or linked covalently, typically containing from 5 to 12 carbon atoms, preferably from 6 to 10 carbon atoms, wherein at least one ring is aromatic. The aromatic ring may optionally include one to two additional rings (either cycloalkyl, heterocycloalkyl or heteroaryl) fused thereto. Aryl is also intended to include the partially hydrogenated derivatives of the carbocyclic systems enumerated herein, as long as at least one ring is aromatic. Non-limiting examples of aryl include phenyl, biphenyl, biphenylenyl, 5- or 6-tetralinyl, naphthalen-1- or -2-yl, 4-, 5-, 6 or 7-indenyl, 1-2-, 3-, 4- or 5-acenaphthylenyl, 3-, 4- or 5-acenaphthenyl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, and 1-, 2-, 3—, 4- or 5-pyrenyl.

“Carboxy” refers to —COOH group.

“Cyano” refers toCN group.

“Cycloalkyl” refers to a cyclic alkyl group, i.e., a monovalent, saturated, or unsaturated hydrocarbyl group having 1 or 2 cyclic structures. Cycloalkyl includes monocyclic or bicyclic hydrocarbyl groups. Cycloalkyl groups may comprise 3 or more carbon atoms in the ring and generally, cycloalkyl groups of this invention comprise from 3 to 10, preferably from 3 to 8 carbon atoms, more preferably from 3 to 6 carbon atoms. This definition of “cycloalkyl” encompasses polycyclic cycloalkyls (e.g., bicycles) and bridged cycloalkyl structures, including cycles bound together through one atom (“spiro”) or through two atoms. Non-limiting examples of cycloalkyl groups include cyclopropyl, cyclobutyl, cyclopropyl, cyclohexyl, cycoheptyl, cyclooctanyl, cyclononanyl, cyclodecanyl, norbornyl, adamantyl, bicyclo[2.2.2]octanyl, bicyclo[4.4.0]decanyl, bicyclo[3.2.1]octanyl, bicyclo[3.3.1]nonanyl, bicyclo[2.1.1]hexane, 2,3-dihydro-1H-indenyl, 1,2,3,4-tetrahydronaphthalenyl, decahydronaphthalenyl, 1,2,3,4-tetrahydronaphthalenyl, and octahydropentalenyl.

“Halogen” or “halo” means fluoro, chloro, bromo, or iodo. Generally, halo groups of this invention are fluoro, chloro or bromo.

“Haloalkyl” refers to an alkyl group wherein one or more hydrogen atoms are replaced with a halogen atom. Non-limiting examples of haloalkyl groups include chloromethyl, 1-bromoethyl, fluoromethyl, difluoromethyl, trifluoromethyl and 1,1,1-trifluoroethyl.

“Haloalkoxy” refers to an alkoxy group wherein one or more hydrogen atoms are replaced with a halogen atom. Non-limiting examples of haloalkoxy groups include chloromethoxy, 1-bromoethoxy, fluoromethoxy, difluoromethoxy, trifluoromethoxy and 1,1,1-trifluoroethoxy.

“Heteroalkyl” refers to an alkyl group wherein one or more carbon atoms are replaced by a heteroatom, for example oxygen, nitrogen or sulfur atom, and wherein the resulting heteroalkyl group comprises at least one carbon atom. In heteroalkyl groups, the heteroatoms are bound along the alkyl chain only to carbon atoms, i.e., each heteroatom is separated from any other heteroatom by at least one carbon atom, typically by at least two carbon atoms. The nitrogen and sulfur heteroatoms may optionally be oxidized and the nitrogen heteroatoms may optionally be quaternized (e.g., sulfur may be oxidized as SO or SO₂). Heteroalkyl groups may further include one or more ═O and/or ═S groups. In one embodiment, at least two carbon atoms are replaced by a heteroatom. In one embodiment, the heteroalkyl is bound to another group or molecule through a carbon atom, i.e., the binding atom is not selected among the heteroatoms included therein. In one embodiment, the heteroalkyl is bound to another group or molecule through one of the heteroatoms included therein. When substituted by one or more other group(s), an heteroalkyl may be substituted either through a carbon atom or through a heteroatom (e.g., nitrogen), unless otherwise specified. Non-limiting examples of heteroalkyl include alkoxy, ethers and polyethers (e.g., polyethylene glycol), secondary and tertiary amines and polyamines, thioethers and polythioethers, and combinations thereof.

“Heterocycloalkyl” refers to a cyclic heteroalkyl group, typically comprising from 2 to 15 carbon atoms, preferably from 2 to 11 carbon atoms, more preferably from 2 to 7 carbon atoms, furthermore preferably from 2 to 6 carbon atoms. Heterocycloalkyl groups are typically 3- to 7-membered, preferably 5- or 6-membered. Heterocycloalkyl are typically monocyclic or bicyclic, preferably monocyclic. This definition encompasses polycyclic heterocycloalkyls (e.g., bicycles) and bridged heterocycloalkyl structures, including cycles bound together through one atom (“spiro”) or through two atoms. In one embodiment, the heterocycloalkyl is bound to another group or molecule through a carbon atom, i.e., the binding atom is not selected among the heteroatoms included therein. In one embodiment, the heterocycloalkyl is bound to another group or molecule through one of the heteroatoms included therein. When substituted by one or more other group(s), an heterocycloalkyl may be substituted either through a carbon atom or through a heteroatom (e.g., nitrogen), unless otherwise specified. Non-limiting examples of heterocycloalkyl include aziridine, pyrrolidine, piperidine, piperazine, morpholine, thiomorpholine, azepane, azocane, octahydro-1H-isoindole, decahydroisoquinoline, tetrahydrofuran, tetrahydropyran, tetrahydroisoquinoline (e.g., 1,2,3,4-tetrahydroisoquiline), hexahydropyridazine, hexahydropyrazine, hexahydropyrimidine, decahydroquinoline, octahydropyrrolo[3,4-c]pyrrole, isoindoline, 1,2,3,4-tetrahydroquinoline and oxetane.

“Hydroxyl” refers toOH group.

“Nitro” refers to —NO₂ group.

“Oxo” refers to ═O group.

“Solvate” refers to a molecular complex comprising a compound of the invention and contains stoichiometric or sub-stoichiometric amounts of one or more pharmaceutically acceptable solvent molecule such as ethanol. The term “hydrate” refers to when said solvent is water.

### General Definitions

The terms “a” and “an” refer to one or to more than one (i.e., to at least one) of the grammatical object of the article. By way of example, “an element” means one element or more than one element.

The term “about” preceding a figure means plus or minus 10%, or less, of the value of said figure. It is to be understood that the value to which the term “about” refers is itself also specifically, and preferably, disclosed.

“Amino acid” refers to both natural and synthetic amino acids, and both D- and L-amino acids. They are represented by their full name, their three-letter code or their one-letter code as well-known in the art. “Standard amino acid” or “naturally occurring amino acid” means any of the twenty standard L-amino acids commonly found in naturally occurring polypeptides. “Non-standard amino acid” means any amino acid, other than the standard amino acids, regardless of whether it is prepared synthetically or derived from a natural source. For example, naphtlylalanine can be substituted for tryptophan to facilitate synthesis. Other synthetic amino acids that can be substituted include, but are not limited to, L-hydroxypropyl, L-3,4-dihydroxyphenylalanyl, alpha-amino acids such as L-alpha-hydroxylysyl and D-alpha-methylalanyl, L-alpha-methylalanyl, beta-amino acids, and isoquinolyl. The polypeptides as described herein may comprise standard amino acids or non-standard amino acids. The term “amino acid” also encompasses chemically modified amino acids, including, but not limited to, salts, amino acid derivatives (such as amides), and substitutions. Amino acids contained within the polypeptides and fusion proteins as described herein, and particularly at the carboxy- or amino-terminus, can thus be modified by methylation, amidation, acetylation or substitution with other chemical groups. Additionally, a disulfide linkage may be present or absent in the polypeptides as described herein. Other polypeptide mimetics encompassed herein include polypeptides or fusion proteins as described herein having the following modifications: i) polypeptides wherein one or more of the peptidyl-C(O) NR-linkages (bonds) have been replaced by a non-peptidyl linkage such as a —CH₂-carbamate linkage (—CH₂OC(O)NR—), a phosphonate linkage, a —CH₂-sulfonamide (—CH₂—S(O)₂NR—) linkage, a urea (—NHC(O)NH—) linkage, a —CH₂-secondary amine linkage, or with an alkylated peptidyl linkage (—C(O) NR—) wherein R is C₁-C₄ alkyl; ii) polypeptides wherein the N-terminus is derivatized to a —NRR¹ group, to a —NRC(O)R group, to a —NRC(O)OR group, to a —NRS(O)₂R group, to a —NHC(O)NHR group, where R and R¹ are hydrogen or C₁-C₄ alkyl with the proviso that R and R¹ are not both hydrogen; iii) polypeptides wherein the C terminus is derivatized to —C(O)R², where R² is selected from the group consisting of C₁-C₄ alkoxy, and —NR³R⁴, where R³ and R⁴ are independently selected from the group consisting of hydrogen and C₁-C₄ alkyl.

“Cell line” refers to cells obtained from the proliferation of a single cell and therefore forming a uniform population of cells comprising an identical genetic material.

“Complement” or “complementation” with reference to the first and second polypeptides as described herein refers to the capacity of said two polypeptides to assemble together to reconstitute a scaffold to which the molecular inducer of proximity as described herein can bind.

The term “CIP system” refers to a chemically induced proximity system.

The term “em” refers to emission, as in emission wavelength.

The term “ex” refers to excitation, as in excitation wavelength.

“Fluorogenic chromophore” or “fluorogen” refer to a chromophore, the brightness of which can be significantly enhanced by an environmental change. A fluorogenic chromophore or fluorogen is substantially non-fluorescent in solution under its free form, but brightens up when placed into an environment constraining its conformation and excluding deexcitation of its excited state by non-radiative pathways. In some embodiments, the fluorogenic chromophore is almost invisible in solution and becomes fluorescent upon binding of a protein scaffold, such as the one formed by the two polypeptides described herein.

“Identity” or “identical”, when used in a relationship between the sequences of two or more polypeptides or of two or more nucleic acids, refers to the degree of sequence relatedness between polypeptides or nucleic acids (respectively), as determined by the number of matches between strings of two or more amino acid residues or of two or more nucleotides, respectively. “Identity” measures the percent of identical matches between the smaller of two or more sequences with gap alignments (if any) addressed by a particular mathematical model or computer program (i.e., “algorithms”). Identity of related polypeptides or nucleic acid sequences can be readily calculated by known methods. Such methods include for example those described in Arthur M. Lesk, Computational Molecular Biology: Sources and Methods for Sequence Analysis (New-York: Oxford University Press, 1988); Douglas W. Smith, Biocomputing: Informatics and Genome Projects (New-York: Academic Press, 1993); Hugh G. Griffin and Annette M. Griffin, Computer Analysis of Sequence Data, Part 1 (New Jersey: Humana Press, 1994); Gunnar von Heinje, Sequence Analysis in Molecular Biology: Treasure Trove or Trivial Pursuit (Academic Press, 1987); Michael Gribskov and John Devereux, Sequence Analysis Primer (New York: M. Stockton Press, 1991); and Carillo et al., 1988. SIAM J. Appl. Math. 48 (5): 1073-1082. Preferred methods for determining identity are designed to give the largest match between the sequences tested. Methods of determining identity are described in publicly available computer programs. Examples of computer program methods for determining identity between two sequences include the GCG program package, including GAP (Devereux et al., 1984. Nucl. Acid. Res. 12 (1 Pt 1): 387-395; Genetics Computer Group, University of Wisconsin Biotechnology Center, Madison, WI), BLASTP, BLASTN, TBLASTN and FASTA (Altschul et al., 1990. J. Mol. Biol. 215 (3): 403-410). The BLASTX program is publicly available from the National Center for Biotechnology Information (NCBI) and other sources (BLAST Manual, Altschul et al. NCB/NLM/NIH Bethesda, Md. 20894; Altschul et al., 1990. J. Mol. Biol. 215 (3): 403-410). The well-known Smith Waterman algorithm may also be used to determine identity.

“Nucleic acid” or “nucleic acid molecule” refers to a polymer of nucleotides covalently linked by phosphodiester bonds, such as deoxyribonucleic acids (DNAs) or ribonucleic acids (RNAs), in either single- or double-stranded form. A used herein, the term “nucleic acid” thus encompasses single-stranded, partially double-stranded, and fully double-stranded nucleic acids. Unless specifically limited, the term encompasses nucleic acids containing known analogues of natural nucleotides that have similar binding properties as the reference nucleic acid and are metabolized in a manner similar to naturally occurring nucleotides.

“Nucleic acid sequence” or “nucleotide sequence” refers to a contiguous sequence of nucleotides in a single nucleic acid. Unless otherwise indicated, a particular nucleic acid sequence also implicitly encompasses conservatively modified variants thereof (e.g., degenerate codon substitutions), alleles, orthologs, SNPs (single-nucleotide polymorphisms), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Unless otherwise specified, a nucleotide sequence encoding an amino acid sequence includes all nucleotide sequences that are degenerate versions of each other and that encode the same amino acid sequence. The nucleotide sequence that encodes a protein or an RNA may also include introns to the extent that the nucleotide sequence encoding the protein or RNA may in some version contain at least one intron. Notably, a particular nucleic acid sequence described herein implicitly comprises its corresponding complementary sequence. It should be noted that a particular nucleic acid sequence described herein implicitly comprises the DNA sequence and the corresponding RNA sequence.

“Pharmaceutically acceptable excipient” or “pharmaceutically acceptable carrier” refers to an excipient or carrier that does not produce an adverse, allergic or other untoward reaction when administered to a subject, such as an animal, in particular a mammal, preferably a human. It includes any and all solvents, such as, for example, dispersion media, coatings, antibacterial and antifungal agents, isotonic and absorption delaying agents. A pharmaceutically acceptable excipient or carrier refers to a non-toxic solid, semi-solid or liquid filler, diluent, encapsulating material or formulation auxiliary of any type. For human administration, preparations should meet sterility, pyrogenicity, general safety and purity standards as required by the regulatory offices such as the FDA (US Food and Drug Administration) or EMA (European Medicines Agency).

“Reporter protein” refers to a protein that may be detected, localized or quantified as a way to indirectly assess a target of interest or a mechanism of interest.

“Sample” refers to a specimen or small quantity of material, in particular of biological material, generally solid or liquid. “Sample” may thus also refer to cells or tissues or organisms of interest.

“Vector” refers to a vehicle by which a nucleotide sequence (e.g., a DNA sequence or an RNA sequence), for example a nucleotide sequence encoding a polypeptide or protein can be introduced into a host cell, so as to transform, transfect or transduce the host cell and promote replication and/or expression (e.g., transcription and/or translation) of the introduced nucleotide sequence.

“Expression vector” refers to a vector comprising regulatory elements (or regulatory sequences) operatively linked to a nucleotide sequence to be expressed. An expression vector thus comprises sufficient cis-acting regulatory elements for controlling the expression of the nucleotide sequence; other elements that may be required for controlling the expression of the nucleotide sequence of interest may be supplied by a host cell.

### DETAILED DESCRIPTION

One object of the invention is a proximity inducing system comprising two polypeptides and a molecular inducer of proximity as described herein, wherein:
a first polypeptide comprises or consists of an amino acid sequence as set forth in SEQ ID NO: 1 or an amino acid sequence having at least about 30% identity with SEQ ID NO: 1 as described herein, or a truncated fragment thereof as described herein; and
a second polypeptide comprises or consists of an amino acid sequence as set forth in SEQ ID NO: 2 or an amino acid sequence having at least about 30% identity with SEQ ID NO: 2 as described herein, or a truncated fragment thereof as described herein.

In some embodiments, the present invention relates to a proximity inducing system comprising two polypeptides and a molecular inducer of proximity as described herein, wherein:
a first polypeptide comprises or consists of an amino acid sequence as set forth in SEQ ID NO: 1 or an amino acid sequence having at least about 30% identity with SEQ ID NO: 1 as described herein, or a truncated fragment thereof as described herein; and
a second polypeptide comprises or consists of an amino acid sequence as set forth in SEQ ID NO: 2 or an amino acid sequence having at least about 30% identity with SEQ ID NO: 2 as described herein, or a truncated fragment thereof as described herein,
   wherein the first polypeptide (or a truncated fragment thereof), the second polypeptide (or a truncated fragment thereof) and the molecular inducer of proximity form a ternary assembly.

In some embodiments, the present invention relates to a proximity inducing system comprising two polypeptides and a molecular inducer of proximity as described herein, wherein:
a first polypeptide comprises or consists of an amino acid sequence as set forth in SEQ ID NO: 1 or an amino acid sequence having at least about 30% identity with SEQ ID NO: 1 as described herein, or a truncated fragment thereof as described herein; and
a second polypeptide comprises or consists of an amino acid sequence as set forth in SEQ ID NO: 2 or an amino acid sequence having at least about 30% identity with SEQ ID NO: 2 as described herein, or a truncated fragment thereof as described herein,
   wherein the first polypeptide (or a truncated fragment thereof), the second polypeptide (or a truncated fragment thereof) and the molecular inducer of proximity form a ternary assembly, with the concentration of molecular inducer of proximity required to reach half maximal assembly being of less than about 5 μM.

As used herein, the expression “the first polypeptide, the second polypeptide and the molecular inducer of proximity form a ternary assembly” encompasses the formation of a ternary assembly between a truncated fragment of the first polypeptide as described herein, a truncated fragment of the second polypeptide as described herein and the molecular inducer of proximity as described herein.

According to the present invention, in presence of a molecular inducer of proximity, the first polypeptide as described herein and the second polypeptide as described herein (or truncated fragments thereof) are able to complement and bind the molecular inducer of proximity as described herein to form a ternary assembly. Strikingly, as illustrated hereinafter in the examples, the presence of the molecular inducer of proximity triggers the complementation of the first and the second polypeptides (or truncated fragments thereof) and their binding to the molecular inducer of proximity independently of any initial proximity between the first and the second polypeptides. In others words, a preexisting proximity between the first and the second polypeptides (or truncated fragments thereof) is not required for the molecular inducer of proximity to be able to trigger the complementation of the first and the second polypeptides (or truncated fragments thereof) and their binding to the molecular inducer of proximity.

According to the present invention, the first polypeptide (or a truncated fragment thereof), the second polypeptide (or a truncated fragment thereof) and the molecular inducer of proximity thus form a ternary assembly independently of any initial proximity between the first and the second polypeptides (or truncated fragments thereof). In some embodiments, the first polypeptide (or a truncated fragment thereof), the second polypeptide (or a truncated fragment thereof) and the molecular inducer of proximity thus form a ternary assembly in the absence of any preexisting proximity between the first and the second polypeptides (or truncated fragments thereof).

In some embodiments, by “proximity” it is meant a distance shorter than about 20 nm, preferably shorter than about 10 nm, and more preferably shorter than about 5 nm.

Accordingly, the first polypeptide as described herein, or a truncated fragment thereof, is not able on its own to bind the molecular inducer of proximity as described herein. Similarly, the second polypeptide as described herein, or a truncated fragment thereof, is not able on its own to bind the molecular inducer of proximity as described herein. Moreover, the first polypeptide as described herein and the second polypeptide as described herein (or truncated fragments thereof) are not able to complement in the absence of the molecular inducer of proximity as described herein.

In some embodiments, the formation of a ternary assembly between the first polypeptide as described herein (or a truncated fragment thereof), the second polypeptide as described herein (or a truncated fragment thereof), and the molecular inducer of proximity as described herein is rapid. In some embodiments, by “rapid”, it is meant that a ternary assembly between the first polypeptide as described herein (or a truncated fragment thereof), the second polypeptide as described herein (or a truncated fragment thereof), and the molecular inducer of proximity as described herein is formed in less than about 90 seconds. In some embodiments, by “rapid”, it is meant that half maximal assembly between the first polypeptide as described herein (or a truncated fragment thereof), the second polypeptide as described herein (or a truncated fragment thereof), and the molecular inducer of proximity as described herein is reached in less than about 90 seconds, preferably in less than about 60 seconds, more preferably in less than about 45 second, even more preferably in less than about 30 seconds.

In some embodiments, the formation of a ternary assembly between the first polypeptide as described herein (or a truncated fragment thereof), the second polypeptide as described herein (or a truncated fragment thereof), and the molecular inducer of proximity as described herein is stable.

In some embodiments, the formation of a ternary assembly between the first polypeptide as described herein (or a truncated fragment thereof), the second polypeptide as described herein (or a truncated fragment thereof), and the molecular inducer of proximity as described herein is reversible.

As used herein, the concentration of molecular inducer of proximity required to reach half maximal assembly (that may also be referred to as half maximal ternary assembly) means the concentration of molecular inducer of proximity required to reach 50% of the ternary assembly formed between the first polypeptide, the second polypeptide and the molecular inducer of proximity.

Methods to evaluate, assess, or quantify the ternary assembly formed between the first polypeptide, the second polypeptide and the molecular inducer of proximity are well-known in the art. Such methods may notably rely on the evaluation, assessment, or quantification of the fluorescence emitted by a fluorogenic molecular inducer of proximity upon binding to the first and second polypeptides, and include spectrofluorimetry, flow cytometry analysis, and fluorescence microscopy (including laser scanning- and spinning-disk based confocal microscopy, multiphoton-microscopy, super-resolution microscopy).

In some embodiments, the concentration of molecular inducer of proximity required to reach half maximal assembly is less than about 5, 4.5, 4, 3.5, 3, 2.5, 2, 1.5, 1, 0.5, or 0.25 μM. In some embodiments, the concentration of molecular inducer of proximity required to reach half maximal assembly ranges from about 0.05 to about 5 μM, preferably from about 0.1 to about 2.5 μM, more preferably from about 0.1 to about 1 μM.

In some embodiments, the first polypeptide as described herein comprises or consists of an amino acid sequence as set forth in SEQ ID NO: 1 or an amino acid sequence having at least about 30%, 35%, 40%, 45%, 50%, or 55%, preferably at least about 60%, 65%, 70%, 75%, 80%, 85%, 90%, 95%, 96%, 97%, 98%, or 99% or more identity with SEQ ID NO: 1. In some embodiments, the first polypeptide as described herein comprises or consists of an amino acid sequence as set forth in SEQ ID NO: 1 or an amino acid sequence having at least about 60%, 65%, 70%, 75%, 80%, 85%, 90%, 95%, 96%, 97%, 98%, or 99% or more identity with SEQ ID NO: 1.

Examples of sequences having at least about 60% identity with SEQ ID NO: 1 include SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 14. In some embodiments, the sequence having at least about 60% identity with SEQ ID NO: 1 is selected from the group comprising or consisting of SEQ ID NO: 3, SEQ ID NO: 4, SEQ ID NO: 5, SEQ ID NO: 6, SEQ ID NO: 7, SEQ ID NO: 9, SEQ ID NO: 10, SEQ ID NO: 11, SEQ ID NO: 12, SEQ ID NO: 13, and SEQ ID NO: 14.

Examples of sequences having at least about 65% identity with SEQ ID NO: 1 include SEQ ID NO: 3, SEQ ID NO: 4, SEQ ID NO: 5, SEQ ID NO: 9, SEQ ID NO: 10, SEQ ID NO: 11, SEQ ID NO: 12, SEQ ID NO: 13, and SEQ ID NO: 14. In some embodiments, the sequence having at least about 65% with SEQ ID NO: 1 is selected from the group comprising or consisting of SEQ ID NO: 3, SEQ ID NO: 4, SEQ ID NO: 5, SEQ ID NO: 9, SEQ ID NO: 10, SEQ ID NO: 11, SEQ ID NO: 12, SEQ ID NO: 13, and SEQ ID NO: 14.

Examples of sequences having at least about 70% identity with SEQ ID NO: 1 include SEQ ID NO: 9, SEQ ID NO: 10, SEQ ID NO: 11, and SEQ ID NO: 12. In some embodiments, the sequence having at least about 70% identity with SEQ ID NO: 1 is selected from the group comprising or consisting of SEQ ID NO: 9, SEQ ID NO: 10, SEQ ID NO: 11, and SEQ ID NO: 12.

In some embodiments, the first polypeptide comprises or consists of an amino acid sequence as set forth in SEQ ID NO: 1. In the experimental section hereinafter, the polypeptide consisting of an amino acid sequence as set forth in SEQ ID NO: 1 is referred to as “^{FIRE}mate”.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein comprises at least one, two, three, four, or five of the following amino acid residues at the indicated positions with reference to SEQ ID NO: 1: a valine (V) at position 30, a leucine (L) at position 41, a threonine (T) at position 72, an alanine (A) at position 83, a threonine (T) at position 95, and/or a leucine (L) at position 109.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein comprises two, three, four, five, or all of the following amino acid residues at the indicated positions with reference to SEQ ID NO: 1: a valine (V) at position 30, a leucine (L) at position 41, a threonine (T) at position 72, an alanine (A) at position 83, a threonine (T) at position 95, and/or a leucine (L) at position 109.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein comprises the following amino acid residues at the indicated positions with reference to SEQ ID NO: 1: a valine (V) at position 30, a leucine (L) at position 41, a threonine (T) at position 72, an alanine (A) at position 83, a threonine (T) at position 95, and a leucine (L) at position 109.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein comprises at least one, two, three, four, five, six, seven, or eight of the following amino acid residues at the indicated positions with reference to SEQ ID NO: 1: an asparagine (N) at position 17, a glutamic acid (E) at position 21, an arginine (R) at position 25, a valine (V) at position 30, a leucine (L) at position 41, a threonine (T) at position 72, an alanine (A) at position 83, a threonine (T) at position 95, and/or a leucine (L) at position 109.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein comprises two, three, four, five, six, seven, eight, or all of the following amino acid residues at the indicated positions with reference to SEQ ID NO: 1: an asparagine (N) at position 17, a glutamic acid (E) at position 21, an arginine (R) at position 25, a valine (V) at position 30, a leucine (L) at position 41, a threonine (T) at position 72, an alanine (A) at position 83, a threonine (T) at position 95, and/or a leucine (L) at position 109.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein comprises the following amino acid residues at the indicated positions with reference to SEQ ID NO: 1: an asparagine (N) at position 17, a glutamic acid (E) at position 21, an arginine (R) at position 25, a valine (V) at position 30, a leucine (L) at position 41, a threonine (T) at position 72, an alanine (A) at position 83, a threonine (T) at position 95, and a leucine (L) at position 109.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein further comprises a glycine (G) at position 69 with reference to SEQ ID NO: 1.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein further comprises at least one, two, three, four, five, or six of the following amino acid residues at the indicated positions with reference to SEQ ID NO: 1: a tryptophan (W) at position 94, an isoleucine (I) at position 96, a proline (P) at position 97, a threonine (T) at position 98, a serine(S) at position 99, an arginine (R) at position 100, and/or a glycine (G) at position 101.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein further comprises the following amino acid residues at the indicated positions with reference to SEQ ID NO: 1: a tryptophan (W) at position 94, an isoleucine (I) at position 96, a proline (P) at position 97, a threonine (T) at position 98, a serine(S) at position 99, an arginine (R) at position 100, and a glycine (G) at position 101.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein comprises:
the following amino acid residues at the indicated positions with reference to SEQ ID NO: 1: a glycine (G) at position 69, a tryptophan (W) at position 94, an isoleucine (I) at position 96, a proline (P) at position 97, a threonine (T) at position 98, a serine(S) at position 99, an arginine (R) at position 100, and a glycine (G) at position 101; and
at least one, two, three, four, or five of the following amino acid residues at the indicated positions with reference to SEQ ID NO: 1: a valine (V) at position 30, a leucine (L) at position 41, a threonine (T) at position 72, an alanine (A) at position 83, a threonine (T) at position 95, and/or a leucine (L) at position 109.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein comprises:
the following amino acid residues at the indicated positions with reference to SEQ ID NO: 1: a glycine (G) at position 69, a tryptophan (W) at position 94, an isoleucine (I) at position 96, a proline (P) at position 97, a threonine (T) at position 98, a serine(S) at position 99, an arginine (R) at position 100, and a glycine (G) at position 101; and
two, three, four, five or all of the following amino acid residues at the indicated positions with reference to SEQ ID NO: 1: a valine (V) at position 30, a leucine (L) at position 41, a threonine (T) at position 72, an alanine (A) at position 83, a threonine (T) at position 95, and/or a leucine (L) at position 109.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein comprises:
the following amino acid residues at the indicated positions with reference to SEQ ID NO: 1: a glycine (G) at position 69, a tryptophan (W) at position 94, an isoleucine (I) at position 96, a proline (P) at position 97, a threonine (T) at position 98, a serine(S) at position 99, an arginine (R) at position 100, and a glycine (G) at position 101; and
at least one, two, three, four, five, six, seven, or eight of the following amino acid residues at the indicated positions with reference to SEQ ID NO: 1: an asparagine (N) at position 17, a glutamic acid (E) at position 21, an arginine (R) at position 25, a valine (V) at position 30, a leucine (L) at position 41, a threonine (T) at position 72, an alanine (A) at position 83, a threonine (T) at position 95, and/or a leucine (L) at position 109.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein comprises:
the following amino acid residues at the indicated positions with reference to SEQ ID NO: 1: a glycine (G) at position 69, a tryptophan (W) at position 94, an isoleucine (I) at position 96, a proline (P) at position 97, a threonine (T) at position 98, a serine(S) at position 99, an arginine (R) at position 100, and a glycine (G) at position 101; and
two, three, four, five, six, seven, eight, or all of the following amino acid residues at the indicated positions with reference to SEQ ID NO: 1: an asparagine (N) at position 17, a glutamic acid (E) at position 21, an arginine (R) at position 25, a valine (V) at position 30, a leucine (L) at position 41, a threonine (T) at position 72, an alanine (A) at position 83, a threonine (T) at position 95, and/or a leucine (L) at position 109.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein comprises at least one, two, three, four, five, six, seven, eight, nine, ten, eleven, twelve, or thirteen of the following amino acid residues at the indicated positions with reference to SEQ ID NO: 1: a valine (V) at position 30, a leucine (L) at position 41, a glycine (G) at position 69, a threonine (T) at position 72, an alanine (A) at position 83, a tryptophan (W) at position 94, a threonine (T) at position 95, an isoleucine (I) at position 96, a proline (P) at position 97, a threonine (T) at position 98, a serine(S) at position 99, an arginine (R) at position 100, a glycine (G) at position 101, and/or a leucine (L) at position 109.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein comprises the following amino acid residues at the indicated positions with reference to SEQ ID NO: 1: a valine (V) at position 30, a leucine (L) at position 41, a glycine (G) at position 69, a threonine (T) at position 72, an alanine (A) at position 83, a tryptophan (W) at position 94, a threonine (T) at position 95, an isoleucine (I) at position 96, a proline (P) at position 97, a threonine (T) at position 98, a serine(S) at position 99, an arginine (R) at position 100, a glycine (G) at position 101, and a leucine (L) at position 109.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein comprises at least one, two, three, four, five, six, seven, eight, nine, ten, eleven, twelve, thirteen, fourteen, fifteen, or sixteen of the following amino acid residues at the indicated positions with reference to SEQ ID NO: 1: an asparagine (N) at position 17, a glutamic acid (E) at position 21, an arginine (R) at position 25, a valine (V) at position 30, a leucine (L) at position 41, a glycine (G) at position 69, a threonine (T) at position 72, an alanine (A) at position 83, a tryptophan (W) at position 94, a threonine (T) at position 95, an isoleucine (I) at position 96, a proline (P) at position 97, a threonine (T) at position 98, a serine(S) at position 99, an arginine (R) at position 100, a glycine (G) at position 101, and/or a leucine (L) at position 109.

In some embodiments, the amino acid sequence having at least about 60%, 65%, 70%, 75%, 80%, 85%, 90%, 95%, 96%, 97%, 98%, or 99% or more identity with SEQ ID NO: 1 as described herein comprises the following amino acid residues at the indicated positions with reference to SEQ ID NO: 1: an asparagine (N) at position 17, a glutamic acid (E) at position 21, an arginine (R) at position 25, a valine (V) at position 30, a leucine (L) at position 41, a glycine (G) at position 69, a threonine (T) at position 72, an alanine (A) at position 83, a tryptophan (W) at position 94, a threonine (T) at position 95, an isoleucine (I) at position 96, a proline (P) at position 97, a threonine (T) at position 98, a serine(S) at position 99, an arginine (R) at position 100, a glycine (G) at position 101, and a leucine (L) at position 109.

In some embodiments, the truncated fragment of the first polypeptide as described herein results from the deletion of consecutive amino acid residues starting from the N-terminal end of the first polypeptide as described herein, preferably the deletion of a number of consecutive amino acid residues ranging from 1 amino acid residue to 40, 35, 30, or 25 amino acid residues from the N-terminal end of the first polypeptide as described herein.

In some embodiments, the truncated fragment of the first polypeptide as described herein is truncated of the amino acid residues from position 1 to at most position 25 of SEQ ID NO: 1 or of the sequence having at least about 60%, 65%, 70%, 75%, 80%, 85%, 90%, 95%, 96%, 97%, 98%, or 99% or more identity with SEQ ID NO: 1 as described herein.

In some embodiments, the truncated fragment of the first polypeptide as described herein is truncated of 1, 2, 3, 4, 5, 6, 7, 8, 9, 10, 11, 12, 13, 14, 15, 16, 17, 18, 19, 20, 21, 22, 23, 24, or 25 consecutive amino acid residue(s) from the N-terminal end (also referred to as N-ter) of SEQ ID NO: 1 or of the sequence having at least about 60%, 65%, 70%, 75%, 80%, 85%, 90%, 95%, 96%, 97%, 98%, or 99% or more identity with SEQ ID NO: 1 as described herein.

In some embodiments, the truncated fragment of the first polypeptide as described herein comprises at least 89, 90, 91, 92, 93, 94, 95, 96, 97, 98, 99, 100, 101, 102, 103, 104, 105, 106, 107, 108, 109, 110, 111, or 112 consecutive amino acid residues from the C-terminal end of SEQ ID NO: 1 or of the sequence having at least about 60%, 65%, 70%, 75%, 80%, 85%, 90%, 95%, 96%, 97%, 98%, or 99% or more identity with SEQ ID NO: 1 as described herein.

In some embodiments, the truncated fragment of the first polypeptide as described herein comprises 89, 90, 91, 92, 93, 94, 95, 96, 97, 98, 99, 100, 101, 102, 103, 104, 105, 106, 107, 108, 109, 110, 111, 112, or 113 consecutive amino acid residues from the C-terminal end of SEQ ID NO: 1 or of the sequence having at least about 60%, 65%, 70%, 75%, 80%, 85%, 90%, 95%, 96%, 97%, 98%, or 99% or more identity with SEQ ID NO: 1 as described herein.

In some embodiments, the second polypeptide comprises or consists of an amino acid sequence as set forth in SEQ ID NO: 2 or an amino acid sequence having at least about 30%, 35%, 40%, 45%, 50%, or 55%, preferably at least about 60%, 65% 70%, 75%, 80%, 85%, 90%, 95%, 96%, 97%, 98%, or 99% or more identity with SEQ ID NO: 2.

In some embodiments, the second polypeptide comprises or consists of an amino acid sequence as set forth in SEQ ID NO: 2 or an amino acid sequence having at least about 60%, 65%, 70%, 75%, 80%, 85%, 90%, 95%, 96%, 97%, 98%, or 99% or more identity with SEQ ID NO: 2.

Examples of sequences having at least about 60% identity with SEQ ID NO: 2 include SEQ ID NO: 15, SEQ ID NO: 16, SEQ ID NO: 17, SEQ ID NO: 18, SEQ ID NO: 19, and SEQ ID NO: 20. In some embodiments, the sequence having at least about 60% identity with SEQ ID NO: 2 is selected from the group comprising or consisting of SEQ ID NO: 15, SEQ ID NO: 16, SEQ ID NO: 17, SEQ ID NO: 18, SEQ ID NO: 19, and SEQ ID NO: 20.

Examples of sequences having at least about 70% identity with SEQ ID NO: 2 include SEQ ID NO: 15, SEQ ID NO: 18, SEQ ID NO: 19, and SEQ ID NO: 20. In some embodiments, the amino acid sequence having at least about 70% identity with SEQ ID NO: 2 is selected from the group comprising or consisting of SEQ ID NO: 15, SEQ ID NO: 18, SEQ ID NO: 19, and SEQ ID NO: 20.

Examples of sequences having at least about 80% identity with SEQ ID NO: 2 include SEQ ID NO: 18, SEQ ID NO: 19, and SEQ ID NO: 20. In some embodiments, the amino acid sequence having at least about 80% identity with SEQ ID NO: 2 is selected from the group comprising or consisting of SEQ ID NO: 18, SEQ ID NO: 19, and SEQ ID NO: 20.

In some embodiments, the second polypeptide comprises or consists of an amino acid sequence as set forth in SEQ ID NO: 2. In the experimental section hereinafter, the polypeptide consisting of an amino acid sequence as set forth in SEQ ID NO: 2 is referred to as “^{FIRE}tag”.

In some embodiments, the amino acid sequence having at least about 60%, 65%, 70%, 75%, 80%, 85%, 90%, 95%, 96%, 97%, 98%, or 99% or more identity with SEQ ID NO: 2 as described herein comprises the following amino acid residue at the indicated position with reference to SEQ ID NO: 2: an arginine (R) at position 3.

In some embodiments, the amino acid sequence having at least about 60%, 65%, 70%, 75%, 80%, 85%, 90%, 95%, 96%, 97%, 98%, or 99% or more identity with SEQ ID NO: 2 as described herein further comprises the following amino acid residue at the indicated position with reference to SEQ ID NO: 2: an isoleucine (I) at position 8.

In some embodiments, the truncated fragment of the second polypeptide as described herein comprises at least 8 consecutive amino acid residues of the second polypeptide as described herein. In some embodiments, the truncated fragment of the second polypeptide as described herein comprises 8, 9, or 10 consecutive amino acid residues of SEQ ID NO: 2 or of the sequence having at least about 60%, 65%, 70%, 75%, 80%, 85%, 90%, 95%, 96%, 97%, 98%, or 99% or more identity with SEQ ID NO: 2 as described herein.

In some embodiments, the truncated fragment of the second polypeptide as described herein results from the deletion of consecutive amino acid residues starting from the N-terminal end and/or the C-terminal end of the second polypeptide as described herein, preferably the deletion of 1, 2 or 3 consecutive amino acid residues.

In some embodiments, the truncated fragment of the second polypeptide as described herein results from the deletion of consecutive amino acid residues starting from the N-terminal end of the second polypeptide as described herein, preferably the deletion of 1, 2 or 3 consecutive amino acid residues. In some embodiments, the truncated fragment of the second polypeptide as described herein results from the deletion of consecutive amino acid residues starting from the C-terminal end of the second polypeptide as described herein, preferably the deletion of 1, 2 or 3 consecutive amino acid residues.

In some embodiments, the truncated fragment of the second polypeptide as described herein comprises at least 8, 9, or 10 consecutive amino acid residues from the N-terminal end of SEQ ID NO: 2 or of the sequence having at least about 60%, 65%, 70%, 75%, 80%, 85%, 90%, 95%, 96%, 97%, 98%, or 99% or more identity with SEQ ID NO: 2 as described herein. In some embodiments, the truncated fragment of the second polypeptide as described herein comprises 8, 9, or 10 consecutive amino acid residues from the N-terminal end of SEQ ID NO: 2 or of the sequence having at least about 60%, 65%, 70%, 75%, 80%, 85%, 90%, 95%, 96%, 97%, 98%, or 99% or more identity with SEQ ID NO: 2 as described herein.

In some embodiments, the second polypeptide as described herein, or a truncated fragment thereof as described herein, is fused to a 113-amino acid fragment of a photoactive yellow protein (PYP) polypeptide having an amino acid sequence as set forth in SEQ ID NO: 21 or an amino acid sequence having at least about 60%, 65%, 70%, 75%, 80%, 85%, 90%, 95%, 96%, 97%, 98%, or 99%, or more identity with the amino acid sequence as set forth in SEQ ID NO: 21, or to a truncated fragment thereof. Said truncated fragment may comprise at least 89, 90, 91, 92, 93, 94, 95, 96, 97, 98, 99, 100, 101, 102, 103, 104, 105, 106, 107, 108, 109, 110, 111, or 112 consecutive amino acid residues from the C-terminal end of SEQ ID NO: 21 or of the sequence having at least about 60, 65%, 70%, 75%, 80%, 85%, 90%, 95%, 96%, 97%, 98%, or 99% or more identity with SEQ ID NO: 21 as described herein. Said truncated fragment may thus comprise 89, 90, 91, 92, 93, 94, 95, 96, 97, 98, 99, 100, 101, 102, 103, 104, 105, 106, 107, 108, 109, 110, 111, or 112 consecutive amino acid residues from the C-terminal end of SEQ ID NO: 21 or of the sequence having at least about 60, 65%, 70%, 75%, 80%, 85%, 90%, 95%, 96%, 97%, 98%, or 99% or more identity with SEQ ID NO: 21 as described herein

In some embodiments, the molecular inducer of proximity is a compound of formula (I)

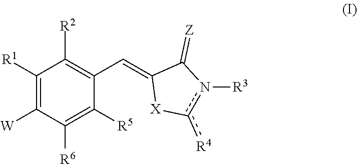

or a salt and/or solvate thereof;
wherein
R¹, R², R⁵ and R⁶ each independently represents H, halo, hydroxyl, aryl, alkyl, cycloalkyl, heteroalkyl (e.g., alkoxy) or heterocycloalkyl,
   wherein said aryl, alkyl, cycloalkyl, heteroalkyl or heterocycloalkyl is optionally substituted by at least one group selected from halo, hydroxyl, oxo, nitro, amido, carboxy, amino, cyano, haloalkoxy and haloalkyl;
R³ represents a non-binding doublet (i.e., a free pair of electrons), H, halo, hydroxyl, aryl, alkyl, cycloalkyl, heteroalkyl or heterocycloalkyl,
   wherein said aryl, alkyl, cycloalkyl, heteroalkyl or heterocycloalkyl is optionally substituted by at least one group selected from halo, hydroxyl, oxo, nitro, amido, carboxy, amino, cyano, haloalkoxy and haloalkyl;
R⁴ represents a single or a double bound, interrupted or terminated by one S, O or N heteroatom, wherein said heteroatom is optionally substituted by at least one group selected from H, hydroxyl, aryl, alkyl, cycloalkyl, heteroalkyl and heterocycloalkyl,
   wherein said aryl, alkyl, cycloalkyl, heteroalkyl or heterocycloalkyl is optionally substituted by at least one group selected from halo, hydroxyl, oxo, nitro, amido, carboxy, amino, cyano, haloalkoxy and haloalkyl;
W represents OH, SH, NHR⁷ or NR⁷R⁸, wherein R⁷ and R⁸ each independently represents H, halo, hydroxyl, aryl, alkyl, cycloalkyl, heteroalkyl or heterocycloalkyl,
   wherein said aryl, alkyl, cycloalkyl, heteroalkyl or heterocycloalkyl is optionally substituted by at least one group selected from halo, hydroxyl, oxo, nitro, amido, carboxy, amino, cyano, haloalkoxy and haloalkyl;
X represents O, S or NH; and
Z represents O, S or NH.

According to the present invention, the alkyl, cycloalkyl, heteroalkyl or heterocycloalkyl groups may be saturated or unsaturated. In some embodiments, the alkyl, cycloalkyl, heteroalkyl or heterocycloalkyl groups are saturated.

In some embodiments, R¹ represents H, alkyl or alkoxy. In some embodiments, R² represents H, alkyl or alkoxy. In some embodiments, R³ represents H. In some embodiments, R⁴ represents a double bound terminated by S, O or NH. In some embodiments, R⁵ represents H In some embodiments, R⁶ represents H, alkyl or alkoxy. In some embodiments, W represents OH. In some embodiments, Z represents O.

In some embodiments, the molecular inducer of proximity is selected from the group comprising or consisting of: (Z)-5-(4-hydroxybenzylidene)-2-thioxo-1,3-thiazolidin-4-one (HBR); (Z)-5-(4-hydroxy-3-methylbenzylidene)-2-thioxo-1,3-thiazolidin-4-one (HMBR); (Z)-5-(4-hydroxy-3,5-dimethoxybenzylidene)-2-thioxothiazolidin-4-one (HBR-3,5DOM); (Z)-5-(4-hydroxy-3-methoxybenzylidene)-2-thioxo-1,3-thiazolidin-4-one (HBR-30M); (Z)-5-(4-hydroxy-3,5-dimethylbenzylidene)-2-thioxothiazolidin-4-one (HBR-3,5DM); (Z)-5-(4-hydroxy-2,5-dimethylbenzylidene)-2-thioxo-1,3-thiazolidin-4-one (HBR-2,5DM); (Z)-5-(3-ethyl-4-hydroxybenzylidene)-2-thioxothiazolidin-4-one (HBR-3E); (Z)-5-(3-ethoxy-4-hydroxybenzylidene)-2-thioxothiazolidin-4-one (HBR-30E); (Z)-5-(4-hydroxybenzylidene)-3-methyl-2-thioxothiazolidin-4-one (HBMR); (Z)-5-(2,4-dihydroxybenzylidene)-2-thioxo-1,3-thiazolidin-4-one (DHBR); (Z)-2-(5-(3-ethyl-4-hydroxybenzylidene)-4-oxo-2-thioxothiazolidin-3-yl) acetic acid (HBRAA-3E); (Z)-2-(5-(4-hydroxy-3-ethoxybenzylidene)-4-oxo-2-thioxothiazolidin-3-yl) acetic acid (HBRAA-30E); (Z)-2-(5-(4-hydroxy-2-methoxybenzylidene)-4-oxo-2-thioxothiazolidin-3-yl) acetic acid (HBRAA-20M); (Z)-2-(5-(4-hydroxybenzylidene)-4-oxo-2-thioxothiazolidin-3-yl) acetic acid (HBRAA); (Z)-2-(5-(4-hydroxy-3-methylbenzylidene)-4-oxo-2-thioxothiazolidin-3-yl) acetic acid (HBRAA-3M); (Z)-2-(5-(4-hydroxy-3-methoxybenzylidene)-4-oxo-2-thioxothiazolidin-3-yl) acetic acid (HBRAA-30M); (Z)-2-(5-(4-hydroxy-2-methoxybenzylidene)-4-oxo-2-thioxothiazolidin-3-yl) acetic acid (HBRAA-20M); (Z)-2-(5-(4-hydroxy-2,5-dimethylbenzylidene)-4-oxo-2-thioxothiazolidin-3-yl) acetic acid (HBRAA-2,5DM); (Z)-2-(5-(4-hydroxy-3,5-dimethylbenzylidene)-4-oxo-2-thioxothiazolidin-3-yl) acetic acid (HBRAA-3,5DM), (Z)-2-(5-(4-hydroxy-3,5-dimethoxybenzylidene)-4-oxo-2-thioxothiazolidin-3-yl) acetic acid (HBRAA-3,5DOM), (Z)-5-(4-hydroxy-3-methylbenzylidene)-4-thioxo-1,3-thiazolidin-2-one (HBIR-3M), and (Z)-5-(4-hydroxy-2,5-dimethoxybenzylidene)-2-thioxo-1,3-thiazolidin-4-one (HBR-2,5DOM).

In some embodiments, the molecular inducer of proximity is a compound of formula (II)

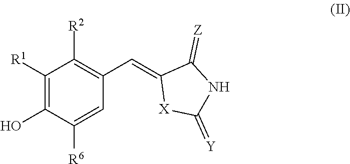

or a salt and/or solvate thereof;
wherein
R¹, R² and R⁶ each independently represents H, alkyl or alkoxy; and
X, Y and Y each independently represents O, S or NH.

In some embodiments, R¹ represents H, methyl (Me) or methoxy (OMe). In some embodiments, R² represents H, methyl or methoxy. In some embodiments, R⁶ represents H, methyl or methoxy.

In some embodiments, R¹, R² and R⁶ respectively represent: (Me, H and H), (H, Me and Me), (Me, H and Me), (OMe, H and OMe) or (H, OMe and OMe).

In some embodiments, X, Y and Y respectively represent: (S, S and O), (S, O and S), (S, NH and O), (S, O and O) or (O, O and O).

In some embodiments, R¹, R², R⁶, X, Y and Y respectively represent: (Me, H, H, S, S and O), (H, Me, Me, S, O and S), (Me, H, Me, S, NH and O), (OMe, H, OMe, S, O and O) or (H, OMe, OMe, O, O and O).

In some embodiments, the molecular inducer of proximity is selected from the group comprising or consisting of (Z)-5-(4-hydroxy-2,5-dimethylbenzylidene)-2-thioxo-1,3-thiazolidin-4-one (HBR-2,5DM), (Z)-5-(4-hydroxy-3-methylbenzylidene)-2-thioxo-1,3-thiazolidin-4-one (HMBR), (Z)-5-(4-hydroxy-3-methylbenzylidene)-4-thioxo-1,3-thiazolidin-2-one (HBIR-3M), (Z)-5-(4-hydroxy-3,5-dimethylbenzylidene)-2-thioxothiazolidin-4-one (HBR-3,5DM), (Z)-5-(4-hydroxy-3,5-dimethoxybenzylidene)-2-thioxothiazolidin-4-one (HBR-3,5DOM), (Z)-5-(4-hydroxy-3-methoxybenzylidene)-2-thioxo-1,3-thiazolidin-4-one (HBR-30M), and (Z)-5-(4-hydroxy-2,5-dimethoxybenzylidene)-2-thioxo-1,3-thiazolidin-4-one (HBR-2,5DOM).

In some embodiments, the molecular inducer of proximity is selected from the group comprising or consisting of (Z)-5-(4-hydroxy-2,5-dimethylbenzylidene)-2-thioxo-1,3-thiazolidin-4-one (HBR-2,5DM), (Z)-5-(4-hydroxy-3-methylbenzylidene)-2-thioxo-1,3-thiazolidin-4-one (HMBR) and (Z)-5-(4-hydroxy-3-methylbenzylidene)-4-thioxo-1,3-thiazolidin-2-one (HBIR-3M).

In some embodiments, the molecular inducer of proximity is (Z)-5-(4-hydroxy-2,5-dimethylbenzylidene)-2-thioxo-1,3-thiazolidin-4-one (HBR-2,5DM)

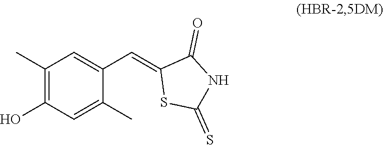 [SMILES: Cc1cc(/C=C2\SC(=S)NC2=O)c(C)cc1O]

In some embodiments, the molecular inducer of proximity is (Z)-5-(4-hydroxy-3-methylbenzylidene)-2-thioxo-1,3-thiazolidin-4-one (HMBR)

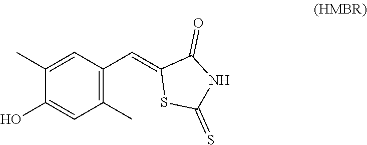 [SMILES: Cc1cc(/C=C2\SC(=S)NC2=O)c(C)cc1O]

In some embodiments, the molecular inducer of proximity is (Z)-5-(4-hydroxy-3-methylbenzylidene)-4-thioxo-1,3-thiazolidin-2-one (HBIR-3M)

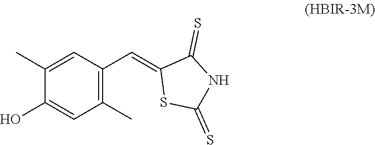 [SMILES: Cc1cc(/C=C2\SC(=S)NC2=S)c(C)cc1O]

In some embodiments, the molecular inducer of proximity is a chromophore.

In some embodiments, the molecular inducer of proximity is non-fluorogenic. In some embodiments, the molecular inducer of proximity is a non-fluorogenic chromophore. Examples of non-fluorogenic molecular inducers of proximity include (Z)-5-(4-hydroxy-3-methylbenzylidene)-4-thioxo-1,3-thiazolidin-2-one (HBIR-3M).

In some embodiments, the molecular inducer of proximity is fluorogenic. In some embodiments, the molecular inducer of proximity is a fluorogenic chromophore. Examples of fluorogenic molecular inducers of proximity include (Z)-5-(4-hydroxy-2,5-dimethylbenzylidene)-2-thioxo-1,3-thiazolidin-4-one (HBR-2,5DM), (Z)-5-(4-hydroxy-3-methylbenzylidene)-2-thioxo-1,3-thiazolidin-4-one (HMBR), (Z)-5-(4-hydroxy-3,5-dimethylbenzylidene)-2-thioxothiazolidin-4-one (HBR-3,5DM), (Z)-5-(4-hydroxy-3,5-dimethoxybenzylidene)-2-thioxothiazolidin-4-one (HBR-3,5DOM), (Z)-5-(4-hydroxy-3-methoxybenzylidene)-2-thioxo-1,3-thiazolidin-4-one (HBR-30M), and (Z)-5-(4-hydroxy-2,5-dimethoxybenzylidene)-2-thioxo-1,3-thiazolidin-4-one (HBR-2,5DOM). In some embodiments, the fluorogenic chromophore is selected from the group comprising or consisting of (Z)-5-(4-hydroxy-2,5-dimethylbenzylidene)-2-thioxo-1,3-thiazolidin-4-one (HBR-2,5DM), (Z)-5-(4-hydroxy-3-methylbenzylidene)-2-thioxo-1,3-thiazolidin-4-one (HMBR), (Z)-5-(4-hydroxy-3,5-dimethylbenzylidene)-2-thioxothiazolidin-4-one (HBR-3,5DM), (Z)-5-(4-hydroxy-3,5-dimethoxybenzylidene)-2-thioxothiazolidin-4-one (HBR-3,5DOM), (Z)-5-(4-hydroxy-3-methoxybenzylidene)-2-thioxo-1,3-thiazolidin-4-one (HBR-30M), and (Z)-5-(4-hydroxy-2,5-dimethoxybenzylidene)-2-thioxo-1,3-thiazolidin-4-one (HBR-2,5DOM). In some embodiments, the fluorogenic chromophore is (Z)-5-(4-hydroxy-2,5-dimethylbenzylidene)-2-thioxo-1,3-thiazolidin-4-one (HBR-2,5DM) and (Z)-5-(4-hydroxy-3-methylbenzylidene)-2-thioxo-1,3-thiazolidin-4-one (HMBR).

Another object of the invention is a biological molecule comprising the first polypeptide as described herein, or a truncated fragment thereof as described herein, and/or the second polypeptide as described herein, or a truncated fragment thereof as described herein.

In some embodiments, the biological molecule comprising the first and/or second polypeptide, or a truncated fragment thereof, is a biological molecule to which the first and/or second polypeptide, or a truncated fragment thereof, is coupled or attached, either covalently or non-covalently.

Methods for non-covalently attaching a peptide or polypeptide to a biological molecule as described herein are well-known and include, for example, chemical coupling. Methods for covalently attaching a peptide or polypeptide to a biological molecule as described herein are well-known and include, for example, chemical coupling or genetic fusion as described herein.

As used herein, the term “biological molecule” encompasses any molecule present in a living organism. Examples of biological molecules include amino acids, polypeptides, proteins, monosaccharides, polysaccharides, nucleotides, nucleic acids, lipids, fatty acids, glycolipids, sterols, vitamins, hormones, neurotransmitters, and metabolites. In particular, the biological molecule may be a nucleic acid or a protein. Examples of nucleic acids include DNA and RNA such as messenger RNA (mRNA), transfer RNA (tRNA). Examples of proteins include transcription factors, enzymes, receptors, immunoreceptors, immunoglobulins, signaling proteins.

In some embodiments, the biological molecule is a protein.

Another object of the invention is thus a fusion protein comprising a protein of interest fused to the first polypeptide as described herein, or a truncated fragment thereof as described herein, and/or the second polypeptide as described herein, or a truncated fragment thereof as described herein. As illustrated in the experimental section hereinafter, the first and second polypeptide, or truncated fragments thereof, may each be expressed in fusion with any protein of interest within a host cell by inserting (for example via transformation or transfection) a nucleotide sequence which encodes both the polypeptide and the protein of interest.

Methods for fusing a peptide or polypeptide to a protein of interest are well-known. Briefly, such methods comprise inserting the nucleotide sequence encoding a protein of interest in frame with the nucleic sequence encoding the peptide(s) or polypeptide(s). The nucleic sequence encoding the protein of interest can be inserted so that the encoded peptide or polypeptide is situated at the N-terminal end of the protein of interest or at the C-terminal end of the protein of interest, or internally, as desired. Additionally, a short nucleic sequence encoding a linker or spacer may be present between the sequences coding for the peptide or polypeptide and for the protein of interest.

The protein of interest may be a natural protein, a chimeric protein resulting from the fusion of various protein domains, or a synthetic protein. The protein of interest may be an intracellular protein, a membrane protein, a cell surface protein present at least in part at the extra membranous surface, or a secreted protein. The protein of interest may be a transcription factor, an enzyme, a receptor, an immunoreceptor, an immunoglobulin, or a signaling protein.

In some embodiments, the protein of interest is a reporter protein. Reporter proteins allow to detect, assess and/or monitor a variation in a sample. Examples of variation that can be detected, assessed and/or monitored using reporter proteins include cell signaling, gene expression, metabolite or analyte concentration, cell-cell interaction, cell movement, cell death, intracellular transport, secretion, cell-cycle phase, and circadian rhythm.

In some embodiments, the fusion protein as described herein comprises at least one additional element other than the first and/or second polypeptide as described herein, or truncated fragments thereof as described herein, and the protein of interest. Example of such additional elements include linkers, targeting signals, localization signals, protease target sites, fluorescent proteins (such as fluorescent protein tags), antibody crystallizable fragments (Fc), enzymes.

In some embodiments, the fusion protein as described herein comprises a linker. Methods for designing or selecting a linker are well-known to one skilled in the art.

Another object of the invention is a kit comprising a biological molecule as described herein, such as a fusion protein as described herein, and a molecular inducer of proximity as described herein.

Another object of the invention is a pair of biological molecules as described herein, wherein one biological molecule comprises the first polypeptide as described herein, or a truncated fragment thereof as described herein, and the other biological molecule comprises the second polypeptide as described herein, or a truncated fragment thereof as described herein.

In some embodiments, the present invention relates to a pair of biological molecules as described herein, with one biological molecule comprising the first polypeptide as described herein, or a truncated fragment thereof as described herein, and the other biological molecule comprising the second polypeptide as described herein, or a truncated fragment thereof as described herein, wherein in presence of a molecular inducer of proximity as described herein, the first polypeptide, the second polypeptide and the molecular inducer of proximity are able to form a ternary assembly, with the concentration of molecular inducer of proximity required to reach half maximal assembly being of less than about 5 μM.

In some embodiments, the pair of biological molecules consists of one biological molecule to which the first polypeptide, or a truncated fragment thereof, is coupled or attached, either covalently or non-covalently; and another biological molecule to which the second polypeptide, or a truncated fragment thereof, is coupled or attached, either covalently or non-covalently.

The pair of biological molecules may consist of two distinct biological molecules. The pair of biological molecules may consist of two biological molecules of different structure, such as a nucleic acid and a protein. The pair of biological molecules may consist of two distinct biological molecules of the same structure, such as two nucleic acids or two proteins. The pair of biological molecules may consist of two copies of the same biological molecule (such as two copies of the same protein), with one copy comprising the first polypeptide, or a truncated fragment thereof, and the other comprising the second polypeptide, or a truncated fragment thereof.

In some embodiment, the pair of biological molecules is a pair of fusion proteins as described herein, with one fusion protein comprising a protein of interest fused to the first polypeptide as described herein, or a truncated fragment thereof as described herein, and the other comprising a protein of interest fused to the second polypeptide as described herein, or a truncated fragment thereof as described herein.

In some embodiments, both fusion proteins of the pair comprise the same protein of interest. In other words, in some embodiments, one fusion protein comprises the first polypeptide, or a truncated fragment thereof, and a protein of interest, and the other fusion protein comprises the second polypeptide, or a truncated fragment thereof, and the same protein of interest. In some embodiments, each fusion protein of the pair comprises a distinct protein of interest. In other words, in some embodiments, one fusion protein comprises the first polypeptide, or a truncated fragment thereof, and a first protein of interest, and the other fusion protein comprises the second polypeptide, or a truncated fragment thereof, and a second, different, protein of interest.

In some embodiments, at least one of the fusion proteins of the pair comprises a protein of interest being a reporter protein. In some embodiments, both fusion proteins of the pair comprise a protein of interest being a reporter protein, either the same reporter protein or different reporter proteins.

In some embodiments, at least one of the fusion proteins of the pair comprises an additional element as described herein, such as a linker, a targeting signal, a localization signal, a protease target site, an antibody crystallizable fragment (Fc), a fluorescent protein (such as a fluorescent protein tag), or an enzyme.

Another object of the invention is a kit comprising a pair of biological molecules as described herein, such as a pair of fusion proteins as described herein, and a molecular inducer of proximity as described herein.

Another object of the invention is a nucleic acid molecule comprising or consisting of the nucleotide sequence encoding the first polypeptide as described herein, or a truncated fragment thereof as described herein, and/or the second polypeptide as described herein, or a truncated fragment thereof as described herein. Examples of nucleic acid molecule include DNA molecules and RNA molecules. Examples of DNA molecules include cDNA molecules (complementary DNA). Examples of RNA molecules include mRNA molecules (messenger RNA).

In some embodiments, by “nucleotide sequence encoding the first and/or the second polypeptide as described herein, or a truncated fragment thereof as described herein” it is meant all nucleotide sequences that are degenerate versions of each other and that encode the same first and/or second polypeptide, or a truncated fragment thereof. One skilled in the art is familiar with methods for adapting a coding sequence on the basis of the genetic code, such as methods making use of codon degeneracy to introduce silent mutations and methods taking into account codon usage bias and variation of the standard genetic code relevant to the host cell considered.

In some embodiments, the nucleic acid molecule comprises or consists of a nucleotide sequence encoding a first polypeptide comprising or consisting of an amino acid sequence as set forth in SEQ ID NO: 1, or a truncated fragment thereof as described herein. An example of nucleotide sequence encoding a first polypeptide comprising or consisting of an amino acid sequence as set forth in SEQ ID NO: 1 is a nucleotide sequence comprising or consisting of the sequence as set forth in SEQ ID NO: 22. Thus, in some embodiments, the nucleic acid molecule comprises or consists of a nucleotide sequence as set forth in SEQ ID NO: 22 or a nucleotide sequence having at least about 50%, 55%, 60%, 65%, 70%, 75%, 80%, 85%, 90%, 95%, 96%, 97%, 98%, or 99% or more identity with SEQ ID NO: 22.

In some embodiments, the nucleic acid molecule comprises or consists of a nucleotide sequence encoding the second polypeptide comprising or consisting of an amino acid sequence as set forth in SEQ ID NO: 2, or a truncated fragment thereof as described herein. An example of nucleotide sequence encoding a second polypeptide comprising or consisting of an amino acid sequence as set forth in SEQ ID NO: 2 is a nucleotide sequence comprising or consisting of as set forth in SEQ ID NO: 23. Thus, in some embodiments, the nucleic acid molecule comprises or consists of a nucleotide sequence as set forth in SEQ ID NO: 23 or a nucleotide sequence having at least about 50%, 55%, 60%, 65%, 70%, 75%, 80%, 85%, 90%, 95%, 96%, 97%, 98%, or 99% or more identity with SEQ ID NO: 23.

In some embodiments, the nucleic acid molecule comprises or consists of a nucleotide sequence encoding a fusion protein as described herein.

Another object of the invention is a kit comprising the nucleic acid molecule as described herein, and a molecular inducer of proximity as described herein.

Another object of the invention is a pair of nucleic acid molecules as described herein, wherein one nucleic acid molecule comprises or consists of a nucleotide sequence encoding the first polypeptide as described herein, or a truncated fragment thereof as described herein, and the other biological molecule comprises or consists of a nucleotide sequence encoding the second polypeptide as described herein, or a truncated fragment thereof as described herein.

In some embodiments, the pair of nucleic acid molecules consists of one nucleic acid molecule comprising or consisting of a nucleotide sequence encoding a first polypeptide comprising or consisting of an amino acid sequence as set forth in SEQ ID NO: 1, or a truncated fragment thereof as described herein; and another nucleic acid molecule comprising or consisting of a nucleotide sequence encoding a second polypeptide comprising or consisting of an amino acid sequence as set forth in SEQ ID NO: 2, or a truncated fragment thereof as described herein.

In some embodiments, the pair of nucleic acid molecules consists of one nucleic acid molecule comprising or consisting of a nucleotide sequence as set forth in SEQ ID NO: 22 or a nucleotide sequence having at least about 50%, 55%, 60%, 65%, 70%, 75%, 80%, 85%, 90%, 95%, 96%, 97%, 98%, or 99% or more identity with SEQ ID NO: 22; and another nucleic acid molecule comprising or consisting of a nucleotide sequence as set forth in SEQ ID NO: 23 or a nucleotide sequence having at least about 50%, 55%, 60%, 65%, 70%, 75%, 80%, 85%, 90%, 95%, 96%, 97%, 98%, or 99% or more identity with SEQ ID NO: 23.

In some embodiments, the pair of nucleic acid molecules consists of two nucleic acid molecules comprising or consisting of nucleotides sequences encoding a fusion protein as described herein, wherein one nucleic acid molecule comprises or consists of a nucleotide sequence encoding a fusion protein comprising the first polypeptide or a truncated fragment thereof as described herein and a protein of interest as described herein, and the other biological molecule comprises or consists of a nucleotide sequence encoding a second fusion protein comprising the second polypeptide or a truncated fragment thereof as described herein and a protein of interest as described herein.

Another object of the invention is a kit comprising the pair of nucleic acid molecule as described herein, and a molecular inducer of proximity as described herein.

Another object of the invention is a vector, in particular an expression vector, comprising a nucleotide sequence encoding the first polypeptide as described herein, or a truncated fragment thereof as described herein, and/or the second polypeptide as described herein, or a truncated fragment thereof as described herein. Examples of vectors include, without being limited to, plasmids, viral vectors, artificial chromosomes, liposomes, and lipid nanoparticles.

In some embodiments, the vector is a nucleic acid molecule as described herein. Example of such vectors include plasmids, artificial chromosomes.

In some embodiments, the vector as described herein comprises:
a first nucleotide sequence encoding the first polypeptide as described herein, or a truncated fragment thereof as described herein; and
a second nucleotide sequence encoding the second polypeptide as described herein, or a truncated fragment thereof as described herein.

In some embodiments, the vector as described herein comprises:
a first nucleotide sequence encoding a first polypeptide comprising or consisting of an amino acid sequence as set forth in SEQ ID NO: 1 or a truncated fragment thereof as described herein; and
a second nucleotide sequence encoding a second polypeptide comprising or consisting of an amino acid sequence as set forth in SEQ ID NO: 2 or a truncated fragment thereof as described herein.

In some embodiments, the vector as described herein comprises:
a first nucleotide sequence comprising or consisting of the sequence as set forth in SEQ ID NO: 22 or a nucleotide sequence having at least about 50%, 55%, 60%, 65%, 70%, 75%, 80%, 85%, 90%, 95%, 96%, 97%, 98%, or 99% or more identity with SEQ ID NO: 22; and
a second nucleotide sequence comprising or consisting of the sequence as set forth in SEQ ID NO: 23 or a nucleotide sequence having at least about 50%, 55%, 60%, 65%, 70%, 75%, 80%, 85%, 90%, 95%, 96%, 97%, 98%, or 99% or more identity with SEQ ID NO: 23.

The vector may comprise the nucleotide sequence(s) required for the fusion of the nucleic sequence encoding a protein of interest in frame with the nucleic sequence encoding the first polypeptide as described herein and/or the second polypeptide as described herein, or a truncated fragment thereof as described herein.

In some embodiments, the vector as described herein comprises a nucleotide sequence encoding at least one fusion protein as described herein.

Another object of the invention is a kit comprising the vector as described herein, and a molecular inducer of proximity as described herein.

Another object of the invention is a pair of vectors as described herein, wherein one vector comprises a nucleotide sequence encoding the first polypeptide as described herein, or a truncated fragment thereof as described herein, and the other vector comprises a nucleotide sequence encoding the second polypeptide as described herein, or a truncated fragment thereof as described herein.

In some embodiments, the pair of vectors consists of one vector comprising a nucleotide sequence encoding a first polypeptide comprising or consisting of an amino acid sequence as set forth in SEQ ID NO: 1, or a truncated fragment thereof as described herein; and another vector comprising a nucleotide sequence encoding a second polypeptide comprising or consisting of an amino acid sequence as set forth in SEQ ID NO: 2, or a truncated fragment thereof as described herein.

In some embodiments, the pair of vectors consists of one vector comprising a nucleotide sequence as set forth in SEQ ID NO: 22 or a nucleotide sequence having at least about 50%, 55%, 60%, 65%, 70%, 75%, 80%, 85%, 90%, 95%, 96%, 97%, 98%, or 99% or more identity with SEQ ID NO: 22; and another vector comprising a nucleotide sequence as set forth in SEQ ID NO: 23 or a nucleotide sequence having at least about 50%, 55%, 60%, 65%, 70%, 75%, 80%, 85%, 90%, 95%, 96%, 97%, 98%, or 99% or more identity with SEQ ID NO: 23.

In some embodiments, the pair of vectors consists of two vectors each comprising a nucleotide sequence encoding a fusion protein as described herein, wherein one vector comprises a nucleotide sequence encoding a fusion protein comprising the first polypeptide or a truncated fragment thereof as described herein and a protein of interest as described herein, and the other vector comprises a nucleotide sequence encoding a second fusion protein comprising the second polypeptide or a truncated fragment thereof as described herein and a protein of interest as described herein.

Another object of the invention is a kit comprising the pair of vectors as described herein, and a molecular inducer of proximity as described herein.

Another object of the invention is a cell or a cell line comprising or expressing the first polypeptide as described herein, or a truncated fragment thereof as described herein, and/or the second polypeptide as described herein, or a truncated fragment thereof as described herein. In some embodiments, the cell or cell line described herein comprises or expresses both the first polypeptide as described herein, or a truncated fragment thereof as described herein, and the second polypeptide as described herein, or a truncated fragment thereof as described herein.

Another object of the invention is a cell or a cell line comprising or expressing a nucleic acid molecule comprising or consisting of the nucleotide sequence encoding the first polypeptide as described herein, or a truncated fragment thereof as described herein, and/or the second polypeptide as described herein, or a truncated fragment thereof as described herein. In some embodiments, the cell or cell line comprises or expresses a pair of nucleic acid molecules as described herein, wherein one nucleic acid molecule comprises or consists of a nucleotide sequence encoding the first polypeptide as described herein, or a truncated fragment thereof as described herein, and the other biological molecule comprises or consists of a nucleotide sequence encoding the second polypeptide as described herein, or a truncated fragment thereof as described herein.

The cell or cell line as described herein may be a genetically modified cell or cell line, that is to say a cell or cell line genetically modified to express the first polypeptide as described herein, or a truncated fragment thereof as described herein, and/or the second polypeptide as described herein, or a truncated fragment thereof as described herein.

The cell or cell line may be a prokaryotic cell or cell line, or a eukaryotic cell or cell line. The cell or cell line may be a bacterium cell or cell line, an archaea cell or cell line, a yeast cell or cell line, a plant cell or cell line, or an animal cell or cell line. The cell or cell line may be a mammal cell or cell line. The cell or cell line may be a human cell or cell line. The cell or cell line may be a primary cell or cell line, in particular a human primary cell or cell line. The cell or cell line may be an immortalized cell or cell line, in particular a human immortalized cell or cell line. The cell or cell line may be an immune cell or cell line, in particular a human immune cell or cell line.

In some embodiments, the cell or cell line as described herein comprises a vector as described herein. In some embodiments, the cell or cell line as described herein comprises a pair of vectors as described herein.

Within the cell or cell line as described herein, the first polypeptide as described herein, or a truncated fragment thereof as described herein, and/or the second polypeptide as described herein, or a truncated fragment thereof as described herein, may be comprised within a biological molecule as described herein. Thus, within the cell or cell line as described herein, the first and/or second polypeptide, or a truncated fragment thereof, may be coupled or attached, either covalently or non-covalently, to a biological molecule.

In some embodiments, the cell or cell line as described herein comprises or expresses at least one biological molecule comprising the first polypeptide as described herein, or a truncated fragment thereof as described herein, and/or the second polypeptide as described herein, or a truncated fragment thereof as described herein.

In some embodiments, the cell or cell line as described herein comprises or expresses at least one fusion protein comprising the first polypeptide as described herein, or a truncated fragment thereof as described herein, and/or the second polypeptide as described herein, or a truncated fragment thereof as described herein.

In some embodiments, the cell or cell line as described herein comprises or expresses a pair of biological molecules as described herein. Thus, in some embodiments, the cell or cell line as described herein comprises or expresses a first biological molecule comprising the first polypeptide as described herein, or a truncated fragment thereof as described herein, and a second biological molecule comprising the second polypeptide as described herein, or a truncated fragment thereof as described herein.

In some embodiments, the cell or cell line as described herein comprises or expresses two biological molecules, wherein:
a first biological molecule is coupled to the first polypeptide as described herein, or a truncated fragment thereof as described herein;
a second biological molecule is coupled to the second polypeptide as described herein, or a truncated fragment thereof as described herein,
   wherein in presence of a molecular inducer of proximity as described herein, the first polypeptide, the second polypeptide and the molecular inducer of proximity are able to form a ternary assembly, with the concentration of molecular inducer of proximity required to reach half maximal assembly being of less than about 5 μM.

In some embodiments, the cell or cell line as described herein comprises or expresses a pair of fusion proteins as described herein. Thus, in some embodiments, the cell or cell line as described herein comprises or expresses a first fusion protein comprising the first polypeptide as described herein, or a truncated fragment thereof as described herein, and a second fusion protein comprising the second polypeptide as described herein, or a truncated fragment thereof as described herein.

In some embodiments, the cell or cell line as described herein comprises or expresses two fusion proteins as described herein, wherein:
a first fusion protein comprises the first polypeptide as described herein, or a truncated fragment thereof as described herein, and a protein of interest as described herein;
a second fusion protein comprises the second polypeptide as described herein, or a truncated fragment thereof as described herein, and a protein of interest as described herein,
   wherein in presence of a molecular inducer of proximity as described herein, the first polypeptide, the second polypeptide and the molecular inducer of proximity are able to form a ternary assembly, with the concentration of molecular inducer of proximity required to reach half maximal assembly being of less than about 5 μM.

Another object of the invention is a kit comprising the cell or cell line as described herein, and a molecular inducer of proximity as described herein.

In some embodiments, at least one of the fusion proteins comprised within or expressed by the cell or cell line as described herein comprises a reporter protein. In some embodiments, both fusion proteins comprised within or expressed by the cell or cell line as described herein comprise a reporter protein.

In some embodiments, the cell or cell line as described herein is a biosensor. By «biosensor», it is meant that the cell or cell line as described herein may be used to assess and/or monitor a target of interest, a physiological mechanism or a biological process of interest.

Examples of targets of interest include analytes or metabolites. In some embodiments, the cell as described herein may thus be used to assess and/or monitor the presence in the cell of an analyte or metabolite.

Examples of physiological mechanisms or biological processes of interest include apoptosis, cell cycle, viral infection, intracellular protein trafficking and transport.

In some embodiments, the cell or cell line as described herein is a biosensor of proximity. By «biosensor of proximity», it is meant that the cell or cell line as described herein may be used to assess and/or monitor the intracellular localization and/or transport of a biological molecule, in particular a protein, such as a cargo protein.

In some embodiments, at least one of the biological molecule comprised within or expressed by the cell or cell line as described herein is a therapeutic effector. In some embodiments, at least one of the biological molecule comprised within or expressed by the cell or cell line as described herein is a fusion protein comprising a protein of interest which is a therapeutic effector. By “therapeutic effector”, it is meant a biological molecule, in particular a protein, able to induce a therapeutic effect upon administration to a subject.

The therapeutic effector may be a protein selected from the group comprising or consisting of transcription factors, enzymes, receptors, immunoreceptors, immunoglobulins, and signaling proteins. The therapeutic effector may be a signaling protein such as, for example, a chimeric antigen receptor (CAR). The therapeutic effector may be a transcription factor.

In some embodiments, the activity and/or function of the therapeutic effector is controlled through chemically induced proximity, chemically induced localization, chemically induced transport, chemically induced dimerization, and/or chemically induced degradation.

In some embodiments, at least one of the biological molecule comprised within or expressed by the cell or cell line as described herein is a safety switch. In some embodiments, at least one of the biological molecule comprised within or expressed by the cell or cell line as described herein is a fusion protein comprising a protein of interest which is a safety switch. By “safety switch”, it is meant a biological molecule, in particular a protein, able to control the activity and/or viability of a cell, for example by controlling gene expression, and/or by inducing cell death (apoptosis).

The safety switch may be a protein selected from the group comprising or consisting of transcription factors and enzymes. The safety switch may be an enzyme such as, for example, a caspase. The safety switch may be a transcription factor.

In some embodiments, the activity and/or function of the safety switch is controlled through chemically induced proximity, chemically induced localization, chemically induced transport, chemically induced dimerization, and/or chemically induced degradation.

Another object of the present invention is a pharmaceutical composition comprising or consisting of any one of:
a biological molecule as described herein,
a pair of biological molecules as described herein,
a nucleic acid molecule as described herein,
a pair of nucleic acid molecules as described herein,
a vector as described herein,
a pair of vectors as described herein, or
a cell or cell line as described herein; and optionally
   at least one pharmaceutically acceptable excipient or pharmaceutically acceptable carrier.

In some embodiments, the pharmaceutical composition as described herein further comprises a molecular inducer of proximity as described herein.

Another object of the present invention is a medicament comprising or consisting of any one of:
a biological molecule as described herein,
a pair of biological molecules as described herein,
a nucleic acid molecule as described herein,
a pair of nucleic acid molecules as described herein,
a vector as described herein,
a pair of vectors as described herein, or
a cell or cell line as described herein.

In some embodiments, the medicament as described herein further comprises a molecular inducer of proximity a described herein.

Another object of the present invention is a kit-of-parts comprising or consisting of:
a first part comprising any one of:
   a biological molecule as described herein,
   a pair of biological molecules as described herein,
   a nucleic acid molecule as described herein,
   a pair of nucleic acid molecules as described herein,
   a vector as described herein,
   a pair of vectors as described herein, or
   a cell or cell line as described herein; and
   a second part comprising a molecular inducer of proximity a described herein.

Another object of the present invention is any one of:
a biological molecule as described herein,
a pair of biological molecules as described herein,
a nucleic acid molecule as described herein,
a pair of nucleic acid molecules as described herein,
a vector as described herein,
a pair of vectors as described herein,
a cell or cell line as described herein,
a kit comprising any one of the above, and a molecular inducer of proximity as described herein,
a pharmaceutical composition as described herein, or
a kit-of-parts as described herein,
   for use as a medicament.

In some embodiments, any one of:
a cell or cell line as described herein,
a pharmaceutical composition as described herein comprising said cell or cell line,
a medicament as described herein comprising said cell or cell line, or
a kit-of-parts as described herein comprising said cell or cell line,
   is for use as a cell therapy or as a cellular therapy.

In some embodiments, any one of:
a cell or cell line as described herein,
a pharmaceutical composition as described herein comprising said cell or cell line,
a medicament as described herein comprising said cell or cell line, or
a kit-of-parts as described herein comprising said cell or cell line,
   is for use for in vivo transplantation in a subject in need thereof.

Another object of the invention is a method for inducing proximity between two biological molecules in a sample, said method comprising the following steps:
obtaining a first biological molecule coupled to the first polypeptide as described herein, or a truncated fragment thereof as described herein;
obtaining a second biological molecule coupled to the second polypeptide as described herein, or a truncated fragment thereof as described herein;
adding a molecular inducer of proximity as described herein to the sample;
   wherein, upon addition of the molecular inducer of proximity, the first polypeptide, the second polypeptide and the molecular inducer of proximity form a ternary assembly, thereby inducing proximity between the first and the second biological molecules present in the sample.

In some embodiments, the concentration of molecular inducer of proximity required to reach half maximal assembly is less than about 5, 4.5, 4, 3.5, 3, 2.5, 2, 1.5, 1, 0.5, or 0.25 μM. In some embodiments, the concentration of molecular inducer of proximity required to reach half maximal assembly ranges from about 0.05 to about 5 μM, preferably from about 0.1 to about 2.5 μM, more preferably from about 0.1 to about 1 μM.

Thus, in some embodiments, the present invention relates to a method for inducing proximity between two biological molecules in a sample, said method comprising the following steps:
obtaining a first biological molecule coupled to the first polypeptide as described herein, or a truncated fragment thereof as described herein;
obtaining a second biological molecule coupled to the second polypeptide as described herein, or a truncated fragment thereof as described herein;
adding a molecular inducer of proximity as described herein to the sample;
   wherein, upon addition of the molecular inducer of proximity, the first polypeptide, the second polypeptide and the molecular inducer of proximity form a ternary assembly, with the concentration of molecular inducer of proximity required to reach half maximal assembly in the sample being of less than about 5 μM,
   thereby inducing proximity between the first and the second biological molecules present in the sample.

In some embodiments, the method is for inducing proximity between two biological molecules in a sample, said method comprising the following steps:
coupling or attaching a first biological molecule to the first polypeptide as described herein, or a truncated fragment thereof as described herein;
coupling or attaching a second biological molecule to the second polypeptide as described herein, or a truncated fragment thereof as described herein;
adding a molecular inducer of proximity as described herein to the sample;
   wherein, upon addition of the molecular inducer of proximity, the first polypeptide, the second polypeptide and the molecular inducer of proximity form a ternary assembly, with the concentration of molecular inducer of proximity required to reach half maximal assembly in the sample being of less than about 5 μM,
   thereby inducing proximity between the first and the second biological molecules present in the sample.

In some embodiments, the method is for inducing proximity between two biological molecules in a sample, said method comprising the following steps:
expressing in the sample a first biological molecule coupled to the first polypeptide as described herein, or a truncated fragment thereof as described herein;
expressing in the sample a second biological molecule coupled to the second polypeptide as described herein, or a truncated fragment thereof as described herein;
adding a molecular inducer of proximity as described herein to the sample;
   wherein, upon addition of the molecular inducer of proximity, the first polypeptide, the second polypeptide and the molecular inducer of proximity form a ternary assembly, with the concentration of molecular inducer of proximity required to reach half maximal assembly in the sample being of less than about 5 μM,
   thereby inducing proximity between the first and the second biological molecules present in the sample.

The two biological molecules may be two distinct biological molecules. The two biological molecules may be two biological molecules of different structure, such as a nucleic acid and a protein. The two biological molecules may be two distinct biological molecules of the same structure, such as two nucleic acids or two proteins. The two biological molecules may be two copies of the same biological molecule (such as two copies of the same protein), with one copy coupled to the first polypeptide as described herein, or a truncated fragment thereof as described herein, and the other copy coupled to the second polypeptide as described herein, or a truncated fragment thereof as described herein.

The biological molecules may be selected from the group comprising or consisting of amino acids, polypeptides, proteins, monosaccharides, polysaccharides, nucleotides, nucleic acids, lipids, fatty acids, glycolipids, sterols, vitamins, hormones, neurotransmitters, and metabolites. In some embodiments, the biological molecules are proteins or nucleic acids. In some embodiments, the biological molecules are proteins.

In some embodiments, the biological molecules coupled to the first or second polypeptide as described herein, or a truncated fragment thereof as described herein, are fusion proteins comprising said first or second polypeptide, or a truncated fragment thereof, and a protein of interest as described herein. The two fusion proteins may comprise the same protein of interest or different proteins of interest.

In some embodiments, the biological molecules are non-covalently coupled to the first or second polypeptide as described herein, or a truncated fragment thereof as described herein. In some embodiments, the biological molecules are covalently coupled to the first or second polypeptide as described herein, or a truncated fragment thereof as described herein. In some embodiments, the biological molecules are proteins, and said proteins are fused to the first or second polypeptide as described herein, or a truncated fragment thereof as described herein.

In some embodiments, the method is for inducing proximity in a sample between a first biological molecule coupled to the first polypeptide as described herein, or a truncated fragment thereof as described herein, and a second biological molecule coupled to the second polypeptide as described herein, or a truncated fragment thereof as described herein, said method comprising:
adding a molecular inducer of proximity as described herein to the sample, wherein, upon addition of the molecular inducer of proximity, the first polypeptide, the second polypeptide and the molecular inducer of proximity form a ternary assembly, with the concentration of molecular inducer of proximity required to reach half maximal assembly in the sample being of less than about 5 μM,
   thereby inducing proximity between the first and the second biological molecules present in the sample.

The sample may be a biological sample. The sample may be an organism. Organisms considered herein are for instance model organisms used in biomedical research such as, for example, bacterium, yeast, fruit fly, nematode, zebrafish, mouse, rat, guinea pig, rabbit, and dog. Other organisms considered herein are plant organisms, such as Arabidopsis thaliana, Tobacco (for example Nicotiana tabacum), wheat (for example Triticum aestivum, Triticum durum, Triticum compactum), rice (for example Oryza sativa). Accordingly, the organism is not a human organism. The sample may be a cell, a cell line, or a cell culture. Examples of cells include, bacteria cells, archaea cells, yeast cells, plant cells, animal cells. Example of animal cells include mammal cells, in particular human cells.

The cells may be primary cells. The cells may be immortalized cells. The cells in the sample may be alive. The cells in the sample may be fixed for microscopy and imaging.

In some embodiments, the organism or the cells in the sample, or a subset thereof, is/are genetically modified to express the first and second polypeptides as described herein, or truncated fragments thereof as described herein, attached to the biological molecules as described herein, preferably proteins of interest.

In some embodiments, the method is a method for inducing proximity between two biological molecules in a cell sample, said method comprising the following steps:
expressing a first biological molecule coupled to the first polypeptide as described herein, or a truncated fragment thereof as described herein, in the cells of the sample;
expressing a second biological molecule coupled to the second polypeptide as described herein, or a truncated fragment thereof as described herein, in the cells of the sample;
adding a molecular inducer of proximity as described herein to the cell sample;
   wherein, upon addition of the molecular inducer of proximity, the first polypeptide, the second polypeptide and the molecular inducer of proximity form a ternary assembly in the cell sample, with the concentration of molecular inducer of proximity required to reach half maximal assembly in the sample being of less than about 5 μM,
   thereby inducing proximity between the first and the second biological molecules expressed in the cells of the sample.

By “in the cell sample” it is meant that the ternary assembly formed between the first polypeptide, the second polypeptide and the molecular inducer of proximity may be intracellular, extracellular or localized at the membrane of the cells. Indeed, the first biological molecule and/or the second biological molecule expressed in the cells of the sample may be secreted by the cells of the sample or may be localized at the membrane of the cells of the sample.

In some embodiments, the method is a method for inducing proximity between two biological molecules in an organism, such as a plant organism or a non-human model organism, said method comprising the following steps:
expressing a first biological molecule coupled to the first polypeptide as described herein, or a truncated fragment thereof as described herein, in the organism;
expressing a second biological molecule coupled to the second polypeptide as described herein, or a truncated fragment thereof as described herein, in the organism; administering a molecular inducer of proximity as described herein to the organism;
   wherein, upon administration of the molecular inducer of proximity, the first polypeptide, the second polypeptide and the molecular inducer of proximity form a ternary assembly, with the concentration of molecular inducer of proximity required to reach half maximal assembly in the sample being of less than about 5 μM,
   thereby inducing proximity between the first and the second biological molecules expressed in the organism.

One skilled in the art is familiar with methods allowing to genetically modify organisms or cells in a culture in order for them to express a biological molecule of interest. Examples of such methods include transfection, electroporation, injection and transgenesis of nucleic acid molecules, such as a nucleic acid molecules as described herein. Said methods also include transplantation, injection and co-culture of cells modified to express proteins of interest.

In some embodiments, the method is for inducing proximity between two biological molecules in a sample, said method comprising the following steps:
fusing a first biological molecule, preferably a protein, to the first polypeptide as described herein, or a truncated fragment thereof as described herein;
fusing a second biological molecule, preferably a protein, to the second polypeptide as described herein, or a truncated fragment thereof as described herein;
expressing both biological molecules in the sample,
adding a molecular inducer of proximity as described herein to the sample;
   wherein, upon addition of the molecular inducer of proximity, the first polypeptide, the second polypeptide and the molecular inducer of proximity form a ternary assembly, with the concentration of molecular inducer of proximity required to reach half maximal assembly in the sample being of less than about 5 μM,
   thereby inducing proximity between the first and the second biological molecules present in the sample.

In some embodiments, in the methods as described herein, the molecular inducer of proximity as described herein is added in the sample at a concentration of less than about 5, 4.5, 4, 3.5, 3, 2.5, 2, 1.5, 1, 0.5, or 0.25 μM. In some embodiments, in the methods as described herein, the molecular inducer of proximity as described herein is added in the sample at a concentration ranging from about 0.05 to about 5 μM, preferably from about 0.1 to about 2.5 μM, more preferably from about 0.1 to about 1 μM.

The molecular inducer of proximity may be non-fluorogenic. The molecular inducer of proximity may be fluorogenic.

The method may thus further comprise a step of detecting and/or visualizing a fluorescence resulting from the formation of the ternary assembly between the first polypeptide, the second polypeptide and a fluorogenic molecular inducer of proximity. The method may thus allow to detect and/or visualize the localization of the ternary assembly formed between the first polypeptide, the second polypeptide and a fluorogenic molecular inducer of proximity.

The method may further comprise a step of monitoring over time a fluorescence resulting from the formation of the ternary assembly between the first polypeptide, the second polypeptide and a fluorogenic molecular inducer of proximity. The method may thus allow to monitor over time the formation of the ternary assembly between the first polypeptide, the second polypeptide and a fluorogenic molecular inducer of proximity.

One skilled in the art is familiar with the techniques available to detect fluorescence in a sample and is able to choose among those depending on the sample, the excitation and emission spectra of the fluorogenic compound(s) and the desired readout. Examples of such techniques include direct observation, fluorescence spectroscopy, flow cytometry, fluorescence microscopy (including laser scanning- and spinning-disk based confocal microscopy, multiphoton-microscopy, super-resolution microscopy), and fluorescence tomography.

The method may further comprise a step of assessing and/or quantifying a fluorescence resulting from the formation of the ternary assembly between the first polypeptide, the second polypeptide and a fluorogenic molecular inducer of proximity. The method may thus allow to assess and/or quantify the formation of the ternary assembly between the first polypeptide, the second polypeptide and a fluorogenic molecular inducer of proximity.

In some embodiments, the method comprises a step of detecting a variation of the fluorescence resulting from the formation of the ternary assembly between the first polypeptide, the second polypeptide and a fluorogenic molecular inducer of proximity. The method may thus comprise a step of detecting an increase or a decrease of the fluorescence resulting from the formation of the ternary assembly between the first polypeptide, the second polypeptide and a fluorogenic molecular inducer of proximity.

In some embodiments, the method further allows to measure the amount of the ternary assembly formed between the first polypeptide, the second polypeptide and a fluorogenic molecular inducer of proximity relative to a reference value by measuring fluorescence intensity. In some embodiments, said reference value corresponds to the fluorescence intensity value in a control sample. In some embodiments, said reference value corresponds to the fluorescence intensity value(s) at a different location(s) and/or time point(s) in the same sample.

Another object of the invention is an assay relying on the induction of proximity between two biological molecules in a sample, said assay comprising the following steps:
obtaining a first biological molecule coupled to the first polypeptide as described herein, or a truncated fragment thereof as described herein;
obtaining a second biological molecule coupled to the second polypeptide as described herein, or a truncated fragment thereof as described herein;
adding a molecular inducer of proximity as described herein to the sample;
   wherein, upon addition of the molecular inducer of proximity, the first polypeptide, the second polypeptide and the molecular inducer of proximity form a ternary assembly, thereby inducing proximity between the first and the second biological molecules present in the sample.

In some embodiments, the concentration of molecular inducer of proximity required to reach half maximal assembly is less than about 5, 4.5, 4, 3.5, 3, 2.5, 2, 1.5, 1, 0.5, or 0.25 μM. In some embodiments, the concentration of molecular inducer of proximity required to reach half maximal assembly ranges from about 0.05 to about 5 μM, preferably from about 0.1 to about 2.5 μM, more preferably from about 0.1 to about 1 μM.

Thus, in some embodiments, the present invention relates to an assay relying on the induction of proximity between two biological molecules in a sample, said assay comprising the following steps:
obtaining a first biological molecule coupled to the first polypeptide as described herein, or a truncated fragment thereof as described herein;
obtaining a second biological molecule coupled to the second polypeptide as described herein, or a truncated fragment thereof as described herein;
adding a molecular inducer of proximity as described herein to the sample;
   wherein, upon addition of the molecular inducer of proximity, the first polypeptide, the second polypeptide and the molecular inducer of proximity form a ternary assembly, with the concentration of molecular inducer of proximity required to reach half maximal assembly in the sample being of less than about 5 μM,
   thereby inducing proximity between the first and the second biological molecules present in the sample.

In some embodiments, the assay comprises the following steps:
coupling or attaching a first biological molecule to the first polypeptide as described herein, or a truncated fragment thereof as described herein;
coupling or attaching a second biological molecule to the second polypeptide as described herein, or a truncated fragment thereof as described herein;
adding a molecular inducer of proximity as described herein to the sample;
   wherein, upon addition of the molecular inducer of proximity, the first polypeptide, the second polypeptide and the molecular inducer of proximity form a ternary assembly, with the concentration of molecular inducer of proximity required to reach half maximal assembly in the sample being of less than about 5 μM,
   thereby inducing proximity between the first and the second biological molecules present in the sample.

In some embodiments, the assay comprises the following steps:
expressing in the sample a first biological molecule coupled to the first polypeptide as described herein, or a truncated fragment thereof as described herein;
expressing in the sample a second biological molecule coupled to the second polypeptide as described herein, or a truncated fragment thereof as described herein;
adding a molecular inducer of proximity as described herein to the sample;
   wherein, upon addition of the molecular inducer of proximity, the first polypeptide, the second polypeptide and the molecular inducer of proximity form a ternary assembly, with the concentration of molecular inducer of proximity required to reach half maximal assembly in the sample being of less than about 5 μM,
   thereby inducing proximity between the first and the second biological molecules present in the sample.

In some embodiments, the assay relies on the induction of proximity in a sample between a first biological molecule coupled to the first polypeptide as described herein, or a truncated fragment thereof as described herein, and a second biological molecule coupled to the second polypeptide as described herein, or a truncated fragment thereof as described herein, and the assay comprises:
adding a molecular inducer of proximity as described herein to the sample,
   wherein, upon addition of the molecular inducer of proximity, the first polypeptide, the second polypeptide and the molecular inducer of proximity form a ternary assembly, with the concentration of molecular inducer of proximity required to reach half maximal assembly in the sample being of less than about 5 μM,
   thereby inducing proximity between the first and the second biological molecules present in the sample.

The assay as described herein may be used for controlling the localization, transport, stability, degradation and/or function of a biological molecule or of a pair of biological molecules as described herein. In particular, the assay as described herein may be used for controlling in a cell the localization, transport, stability, degradation and/or function of a protein or of a pair of proteins as described herein. As mentioned herein, the protein(s) may be fusion protein(s) comprising the first or second polypeptide as described herein, or a truncated fragment as described herein, and a protein of interest as described herein.

In some embodiments, the assay as described herein is for controlling the localization, transport and/or function of a biological molecule or of a pair of biological molecules.

In some embodiments, the assay as described herein is for controlling the localization of a biological molecule, in particular a protein, or of a pair of biological molecules, in particular a pair of proteins. For example, as illustrated in the experimental section hereinafter, the assay may be used for controlling in a cell the localization of a cytosolic protein, in particular a cytosolic fusion protein, by inducing proximity between said protein and a protein, in particular a fusion protein, localized in a cellular compartment, such as for example an organelle.

In some embodiments, the assay as described herein is for controlling the transport of a biological molecule, in particular a protein, or of a pair of biological molecules, in particular a pair of proteins. For example, as illustrated in the experimental section hereinafter, the assay may be used for controlling the nucleocytoplasmic trafficking of a protein in a cell by inducing proximity between a protein, in particular a fusion protein, comprising a nuclear localization signal (NLS) and a cytosolic protein, in particular a cytosolic fusion protein, such as a protein comprising a nuclear export signal (NES).

In some embodiments, the assay as described herein is for controlling the function of a biological molecule, in particular a protein, or of a pair of biological molecules, in particular a pair of proteins.

In some embodiments, the assay as described herein is for controlling the stability of a biological molecule, in particular a protein, or of a pair of biological molecules, in particular a pair of proteins. In some embodiments, the assay as described herein is for inducing the degradation of a biological molecule, in particular a protein, or of a pair of biological molecules, in particular a pair of proteins.

In some embodiments, the assay as described herein is for detecting in the sample the co-occurrence of two biological molecules, in particular of two proteins. For example, the assay may be used for detecting in a cell the co-occurrence of two biological molecules, in particular of two proteins, in a cellular compartment, such as an organelle.

In some embodiments, in the assays as described herein, the molecular inducer of proximity as described herein is added to the sample at a concentration of less than about 5, 4.5, 4, 3.5, 3, 2.5, 2, 1.5, 1, 0.5, or 0.25 μM. In some embodiments, in the assays as described herein, the molecular inducer of proximity as described herein is added in the sample at a concentration ranging from about 0.05 to about 5 μM, preferably from about 0.1 to about 2.5 μM, more preferably from about 0.1 to about 1 μM.

The molecular inducer of proximity may be non-fluorogenic. The molecular inducer of proximity may be fluorogenic.

The assay may further comprise a step of detecting and/or visualizing a fluorescence resulting from the formation of the ternary assembly between the first polypeptide, the second polypeptide and a fluorogenic molecular inducer of proximity. The assay may thus allow to detect and/or visualize the localization of the ternary assembly formed between the first polypeptide, the second polypeptide and a fluorogenic molecular inducer of proximity.

The assay may further comprise a step of monitoring over time a fluorescence resulting from the formation of the ternary assembly between the first polypeptide, the second polypeptide and a fluorogenic molecular inducer of proximity. The assay may thus allow to monitor over time the formation of the ternary assembly between the first polypeptide, the second polypeptide and a fluorogenic molecular inducer of proximity.

The assay may further comprise a step of assessing and/or quantifying a fluorescence resulting from the formation of the ternary assembly between the first polypeptide, the second polypeptide and a fluorogenic molecular inducer of proximity. The assay may thus allow to assess and/or quantify the formation of the ternary assembly between the first polypeptide, the second polypeptide and a fluorogenic molecular inducer of proximity.

In some embodiments, the assay comprises a step of detecting a variation of the fluorescence resulting from the formation of the ternary assembly between the first polypeptide, the second polypeptide and a fluorogenic molecular inducer of proximity. The assay may thus comprise a step of detecting an increase or a decrease of the fluorescence resulting from the formation of the ternary assembly between the first polypeptide, the second polypeptide and a fluorogenic molecular inducer of proximity.

In some embodiments, the assay further comprises measuring the amount of the ternary assembly formed between the first polypeptide, the second polypeptide and a fluorogenic molecular inducer of proximity relative to a reference value by measuring fluorescence intensity. In some embodiments, said reference value corresponds to the fluorescence intensity value in a control sample. In some embodiments, said reference value corresponds to the fluorescence intensity value(s) at a different location(s) and/or time point(s) in the same sample.

TABLE OF SEQUENCES

| SEQ | | |
|---|---|---|
| ID | Sequence | |
| NO | function | Sequence |
| 1 | synthetic | MEHVAFGSEDIENTLANMDDEQLDRLAFGVIQLDGDGNI |
| | polypeptide | LLYNAAEGDITGRDPKQVIGKNFFKDVAPGTDTPEFYGK |
| | | FKEGAASGNLNTMFEWTIPTSRGPTKVKVHLKKALS |
| 2 | synthetic | GDRYWVFVKRV |
| | polypeptide | |
| 3 | synthetic | METVRFGGDDIENSLAKMDDKKLDELAFGAIQLDANGKI |
| | polypeptide | IQYNAAEGGITGRDPKSVIGKNFFTEVAPCTQSKEFQGRF |
| | | KEGVSSGELNTMFEYVFDYQMTPTKVKVHMKKAIS |
| 4 | synthetic | METVRFGGDDIENALANMDDKKLDTLAFGAIQLDANGKI |
| | polypeptide | IQYNAAEGGITGRDPKSVIGKNFFTDVAPCTQSKEFQGRF |
| | | KEGVKNGDLNTMFEYVFDYQMTPTKVKVHMKKALS |
| 5 | synthetic | METVRFGGDDIENSLAKMDDKALDKLAFGAIQLDGNGKI |
| | polypeptide | IHYNAAEGTITGRDPKTVIGKNFFTDVAPCTQSKEFQGRF |
| | | KEGVQKGDLNTMFEYVFDYQMKPTKVKVHMKKAMT |
| 6 | synthetic | MEIVQFGSDDIENTLSKMSDDKLNDIAFGAIQLDASGKIIQ |
| | polypeptide | YNAAEGDITGRDPGAVVGKNFFNEVAPCTNSPEFKGRFD |
| | | EGVKNGNLNTMFEYVFDYEMQPTKVKVHMKKALT |
| 7 | synthetic | MELLSFGADNIENSLAKMSKGDLNKLAFGAIQLNAQGKI |
| | polypeptide | LQYNAAEGDITGRKPTEVIGKNFFLEVAPCTNRTEFKGRF |
| | | DQGIKSGNLNTMFEYTFDYEMKPTKVKVHMKKALV |
| 8 | synthetic | MEMIKFGQDDIENAMADMGDAQIDDLAFGAIQLDETGTI |
| | polypeptide | LAYNAAEGELTGRSPQDVIGKNFFKDIAPCTDTEEFGGRF |
| | | REGVANGDLNAMFEYVFDYQMQPTKVKVHMKRAIT |
| 9 | synthetic | METVRFGGDDIENSLAKMDDKKLDELAFGAIQLDANGKI |
| | polypeptide | IQYNAAEGGITGRDPKSVIGKNFFTEVAPGTQSKEFQGRF |
| | | KEGVSSGELNTMFEWMIPTSRGPTKVKVHMKKAIS |
| 10 | synthetic | METVRFGGDDIENALANMDDKKLDTLAFGAIQLDANGKI |
| | polypeptide | IQYNAAEGGITGRDPKSVIGKNFFTDVAPGTQSKEFQGRF |
| | | KEGVKNGDLNTMFEWMIPTSRGPTKVKVHMKKALS |
| 11 | synthetic | METVRFGGDDIENSLAKMDDKALDKLAFGAIQLDGNGKI |
| | polypeptide | IHYNAAEGTITGRDPKTVIGKNFFTDVAPGTQSKEFQGRF |
| | | KEGVQKGDLNTMFEWMIPTSRGPTKVKVHMKKAMT |
| 12 | synthetic | MEIVQFGSDDIENTLSKMSDDKLNDIAFGAIQLDASGKIIQ |
| | polypeptide | YNAAEGDITGRDPGAVVGKNFFNEVAPGTNSPEFKGRFD |
| | | EGVKNGNLNTMFEWMIPTSRGPTKVKVHMKKALT |
| 13 | synthetic | MELLSFGADNIENSLAKMSKGDLNKLAFGAIQLNAQGKI |
| | polypeptide | LQYNAAEGDITGRKPTEVIGKNFFLEVAPGTNRTEFKGRF |
| | | DQGIKSGNLNTMFEWMIPTSRGPTKVKVHMKKALV |
| 14 | synthetic | MEMIKFGQDDIENAMADMGDAQIDDLAFGAIQLDETGTI |
| | polypeptide | LAYNAAEGELTGRSPQDVIGKNFFKDIAPGTDTEEFGGRF |
| | | REGVANGDLNAMFEWMIPTSRGPTKVKVHMKRAIT |
| 15 | synthetic | GDTYWIFVKRL |
| | polypeptide | |
| 16 | synthetic | GDTFWIFVKRL |
| | polypeptide | |
| 17 | synthetic | GDSFWIFVKRL |
| | polypeptide | |
| 18 | synthetic | GDTYWVFVKRL |
| | polypeptide | |
| 19 | synthetic | DDTYWVFVKRV |
| | polypeptide | |
| 20 | synthetic | GDSYWIFVKRV |
| | polypeptide | |
| 21 | synthetic | EHVAFGSEDIENTLAKMDDGQLDGLAFGAIQLDGDGNIL |
| | polypeptide | QYNAAEGDITGRDPKQVIGKNFFKDVAPGTDSPEFYGKF |
| | | KEGVASGNLNTMFEYTFDYQMTPTKVKVHMKKALS |
| 22 | Synthetic DNA or | atggagcatgttgcctttggcagtgaggacatcgagaacactctggccaatatggacgacg |
| | RNA | aacaactggataggttggcctttggcgtaattcagctcgatggtgacgggaatatcctgctgt |
| | | acaatgctgctgaaggggacatcactggcagagatcccaaacaggtgattgggaagaact |
| | | tcttcaaggatgttgcacctggaacggatactcccgagttttacggcaaattcaaggaaggc |
| | | gcagcgtcagggaatctgaacaccatgttcgaatggacgataccgacaagcaggggacc |
| | | aaccaaggtcaaggtgcacttgaagaaagccctttcc |
| 23 | Synthetic DNA or | ggtgacagatattgggtctttgtgaaacgggtg |
| | RNA | |

### BRIEF DESCRIPTION OF THE DRAWINGS

FIG. 1 is a schematic representation of the working principle of the proximity inducing system as described herein. The system relies on the coupling (for example through genetic fusion) of two proteins to two distinct polypeptides, called FIREmate (i.e., polypeptide of SEQ ID NO: 1) and ^{FIRE}tag (i.e., polypeptide of SEQ ID NO: 2). These two polypeptides can interact in a specific manner only in presence of a small synthetic molecule (i.e., a molecular inducer of proximity called the “match”) that stabilizes the interaction between the two polypeptides. Advantageously, the match may be a fluorogenic chromophore, such as HBR-2,5DM or HMBR, that becomes fluorescent (i.e., “bright match” also referred to as “FIRE fluorescence” or “FIRE signal”) specifically upon formation of the ternary assembly consisting of the two polypeptides and the match (so-called “FIRE” ternary assembly). The system described herein is thus able to induce proximity between any two proteins and, optionally, to visualize the recruitment process by fluorescence imaging.

FIGS. 2A and 2B are graphs illustrating the fluorogen-dependent interaction between the two polypeptides ^{FIRE}mate and ^{FIRE}tag. The graphs show the normalized average fluorescence detected from about 50,000 HEK293T cells co-expressing the FK506-binding protein (FKBP) fused to ^{FIRE}tag and the FKBP-rapamycin-binding domain of mammalian target of rapamycin (FRB) fused to ^{FIRE}mate treated without (triangles) or with (squares) 500 nM of rapamycin, and with 1, 5, 10, 25 or 50 μM of HBR-2,5DM (2A) or HMBR (2B). Data represent the mean values±SD of three independent experiments.

FIGS. 3A-E illustrate the recruitment of cytoplasmic proteins to mitochondria with the proximity inducing system described herein. FIG. 3A is a schematic representation of the experimental design wherein HeLa cells co-expressing mCherry-^{FIRE}tag and Tom20-ECFP-^{FIRE}mate were treated with 10 μM of match (either HBR-2,5DM or HMBR), and imaged by time-lapse confocal microscopy. FIGS. 3B-C are graphs showing the temporal evolution of the FIRE signal detected upon addition at 0 min of HBR-2,5DM (match1-3B) or HMBR (match2-3C). Data represents the mean values±SD of 20 cells from n=3 independent experiments (3B, D), or of 10 cells from n=3 independent experiments (3C, E). FIGS. 3D-E are sets of representative confocal micrographs of cells before (0 min) and after (5 min) addition of HBR-2,5DM (match1-3D) or HMBR (match2-3E) (mCherry: ex/em 561/606-675 nm; FIRE: ex/em 488/508-570 nm; ECFP: ex/em 445/455-499 nm). Experiments were repeated 3 times with similar results. Scale bars are 20 μm.

FIGS. 4A-D illustrate the recruitment over time of cytoplasmic proteins to mitochondria with the proximity inducing system described herein. (4A, C) HeLa cells co-expressing mCherry-^{FIRE}tag and Tom20-ECFP-^{FIRE}mate were treated with 10 μM HBR-2,5DM and imaged by time-lapse confocal microscopy at 1 image per 5 seconds. (4B, D) U2OS cells co-expressing mCherry-^{FIRE}tag and Tom20-ECFP-^{FIRE}mate were treated with 10 μM HBR-2,5DM and imaged by time-lapse confocal microscopy at 1 image every 2 minutes. FIG. 4A is a representative time-lapse of the HeLa cells from the addition of HBR-2,5DM at 0 sec (ex/em 488/508-570 nm). Experiment was repeated 3 times with similar results. Scale bars are 20 μm. FIG. 4B is a representative time-lapse of the U2OS cells from the addition of HBR-2,5DM at 0 min (mCherry: ex/em 561/606-675 nm; FIRE: ex/em 488/508-570 nm; ECFP: ex/em 445/455-499 nm). Experiment was repeated 3 times with similar results. Scale bars are 20 μm. FIG. 4C is a graph showing the temporal evolution of the FIRE signal detected in HeLa cells upon addition of HBR-2,5DM at 0 sec. Data represents the mean values±SD of 15 cells from n=3 independent experiments. FIG. 4D is a graph showing the temporal evolution of the FIRE signal detected in U2OS cells upon addition of HBR-2,5DM at 0 min. Data represents the mean values±SD of 10 cells from n=3 independent experiments.

FIGS. 5A-D demonstrate that the ^{FIRE}tag positioning does not influence the proximity inducing system described herein. HeLa cells co-expressing either ^{FIRE}tag-mCherry (5A,C) or mCherry-^{FIRE}tag-mCherry (5B, D) and Tom20-ECFP-^{FIRE}mate were treated with 10 μM HBR-2,5DM (match1) and imaged by time-lapse confocal microscopy. FIGS. 5A-B are sets of representative confocal micrographs of cells before (0 min) and after (5 min) addition of HBR-2,5DM (match1) (mCherry: ex/em 561/606-675 nm; FIRE: ex/em 488/508-570 nm; ECFP: ex/em 445/455-499 nm). Experiments were repeated 3 times with similar results. Scale bars are 20 μm. FIGS. 5C-D are graphs showing the temporal evolution of the FIRE signal detected upon addition of HBR-2,5DM at 0 min. Data represents the mean values±SD of 17 cells from n=3 independent experiments (5A, C) or of 13 cells from n=3 independent experiments (5B, D).

FIGS. 6A-E illustrate the recruitment of cytoplasmic proteins to the Golgi apparatus with the proximity inducing system described herein. FIG. 6A is a representation of the experimental design wherein HeLa cells co-expressing mCherry-^{FIRE}tag and ^{FIRE}mate-ECFP-Giantin were treated with 10 UM of match (either HBR-2,5DM or HMBR), and imaged by time-lapse confocal microscopy. FIGS. 6B-C are graphs showing the temporal evolution of the FIRE signal detected upon addition at 0 min of HBR-2,5DM (match1-6B) or HMBR (match2-6C). Data represents the mean values±SD of 10 cells from n=3 independent experiments (6B, D), or of 12 cells from n=3 independent experiments (6C, E). FIGS. 6D-E are sets of representative confocal micrographs of cells before (0 min) and after (5 min) addition of HBR-2,5DM (match1-6D) or HMBR (match2-6E) (mCherry: ex/em 561/606-675 nm; FIRE: ex/em 488/508-570 nm; ECFP: ex/em 445/455-499 nm). Experiments were repeated 3 times with similar results. Scale bars are 20 μm.

FIGS. 7A-E illustrate the recruitment of cytoplasmic proteins to the endoplasmic reticulum with the proximity inducing system described herein. FIG. 7A is a representation of the experimental design wherein HeLa cells co-expressing mCherry-^{FIRE}tag and ^{FIRE}mate-ECFP-Cb5 were treated with 10 μM of match (either HBR-2,5DM or HMBR), and imaged by time-lapse confocal microscopy. FIGS. 7B-C are graphs showing the temporal evolution of the FIRE signal detected upon addition at 0 min of HBR-2,5DM (match1-7C) or HMBR (match2-7D). Data represents the mean values±SD of 17 cells from n=3 independent experiments (7B, D), or of 18 cells from n=3 independent experiments (7C, E). FIGS. 7D-E are sets of representative confocal micrographs of cells before (0 min) and after (5 min) addition of HBR-2,5DM (match1-7D) or HMBR (match2-7E) (mCherry: ex/em 561/606-675 nm; FIRE: ex/em 488/508-570 nm; ECFP: ex/em 445/455-499 nm). Experiments were repeated 3 times with similar results. Scale bars are 20 μm.

FIGS. 8A-E illustrate the recruitment of cytoplasmic proteins to the plasma membrane with the proximity inducing system described herein. FIG. 8A is a representation of the experimental design wherein HEK293 cells co-expressing mCherry-^{FIRE}tag and Lyn11-ECFP-^{FIRE}mate were treated with 10 μM of match (either HBR-2,5DM or HMBR), and imaged by time-lapse confocal microscopy. FIGS. 8B-C are graphs showing the temporal evolution of the FIRE signal detected upon addition at 0 min of HBR-2,5DM (match1-8B) or HMBR (match2-8C). Data represents the mean values±SD of 22 cells from n=3 independent experiments (8B, D), or of 30 cells from n=3 independent experiments (8C, E). FIGS. 8D-E are sets of representative confocal micrographs of cells before (0 min) and after (5 min) addition of HBR-2,5DM (match1-8D) or HMBR (match2-8E) (mCherry: ex/em 561/606-675 nm; FIRE: ex/em 488/508-570 nm; ECFP: ex/em 445/455-499 nm). Experiments were repeated 3 times with similar results. Scale bars are 20 μm.

FIGS. 9A-D demonstrate the reversibility of the proximity inducing system described herein and the possibility to induce non-fluorogenic recruitment. HeLa cells co-expressing mCherry-^{FIRE}tag and ^{FIRE}mate-ECFP-Giantin were treated with 10 μM HBR-2,5DM (match1), washed with HBR-2,5DM-free medium and then treated with 10 μM HBIR-3M. Cells were imaged by time-lapse spinning-disk confocal microscopy. FIGS. 9A-D are sets of representative confocal micrographs of untreated cells (9A), cells after addition of HBR-2,5DM (match1) (9B), cells after washout of HBR-2,5DM (match1) (9C), and cells after addition of HBIR-3M (9D). (mCherry: ex/em 561/606-675 nm; FIRE: ex/em 488/508-570 nm). Scale bars are 10 μm.

FIG. 10 is a graph illustrating the fluorogen-dependent interaction between caged ^{FIRE}tag and ^{FIRE}mate. The graph shows the normalized average fluorescence detected from about 50,000 HEK293T cells co-expressing the FK506-binding protein (FKBP) fused to caged ^{FIRE}tag and the FKBP-rapamycin-binding domain of mammalian target of rapamycin (FRB) fused to ^{FIRE}mate treated without or with 500 nM of rapamycin, and with 1, 5, 10, 25 or 50 μM of HBR-2,5DM. Data represent the mean values±SD of 3 independent experiments.

FIGS. 11A-C illustrate the use of a caged proximity inducing system as described herein. FIG. 11A is a representation of the experimental design wherein HeLa cells co-expressing mCherry-caged^{FIRE}tag and ^{FIRE}mate-ECFP-Giantin were treated with 10 μM of match (i.e., HBR-2,5DM), and imaged by time-lapse confocal microscopy. FIG. 11B is a graph showing the temporal evolution of the FIRE signal detected upon addition of HBR-2,5DM at 0 min, either with ^{FIRE}tag or with caged^{FIRE}tag. Data represents the mean values±SD of 7 cells from n=3 independent experiments. FIG. 11C is a set of representative confocal micrographs of cells before (0 min) and after (9 min) addition of HBR-2,5DM (match1) (mCherry: ex/em 561/606-675 nm; FIRE: ex/em 488/508-570 nm; ECFP: ex/em 445/455-499 nm). Experiments were repeated 3 times with similar results. Scale bars are 20 μm.

FIGS. 12A-B demonstrate that the proximity inducing system described herein is orthogonal to the FRB/FKBP/rapamycin CIP system. FIG. 12A is a representation of the experimental design wherein HeLa cells co-expressing mCherry-FKBP-^{FIRE}tag, TOM20-^{FIRE}mate and FRB-ECFP-Giantin were treated with 10 μM of match (i.e., HBR-2,5DM) and 500 nM rapamycin, and imaged by time-lapse confocal microscopy. FIG. 12B is a set of representative confocal micrographs of cells before and after addition of HBR-2,5DM (match1) and rapamycin (rap) (mCherry: ex/em 561/606-675 nm; FIRE: ex/em 488/508-570 nm; ECFP: ex/em 445/455-499 nm). Scale bars are 20 μm.

FIGS. 13A-D illustrate the nuclear export of proteins mediated by the proximity inducing system described herein. FIG. 13A is a representation of the experimental design wherein HeLa cell co-expressing NLS-mCherry-FIR_^{FIRE}mate and NES-ECFP-^{FIRE}tag were treated with 10 μM of match (i.e., HBR-2,5DM) and imaged by time-lapse confocal microscopy. FIG. 13B is a graph showing the temporal evolution of the ratio nucleus-to-cytoplasm mCherry fluorescence intensities of n=5 cells from 2 independent experiments. FIG. 13C is a graph showing the temporal evolution of the FIRE signal detected upon addition of HBR-2,5DM at 0 min of n=5 cells from 2 independent experiments. FIG. 13D is a set of representative confocal micrographs before (0 min) and after (4 min) treatment with HBR-2,5DM (mCherry: ex/em 561/606-675 nm; FIRE: ex/em 488/508-570 nm; ECFP: ex/em 445/455-499 nm). Scale bars are 20 μm.

### EXAMPLES

The present invention is further illustrated by the following examples.

### Example

### Materials and Methods

### Molecular Cloning

Synthetic oligonucleotides used for cloning were purchased from Integrated DNA technology. PCR reactions were performed with Q5 polymerase (New England Biolabs) in the buffer provided. PCR products were purified using QIAquick PCR purification kit (Qiagen). DNAse I, T4 ligase, Fusion polymerase, Taq ligase and Taq exonuclease were purchased from New England Biolabs and used with accompanying buffers and according to the manufacturer's protocols. Isothermal assemblies (Gibson assembly) were performed using homemade mix prepared according to previously described protocols (Gibson et al. Enzymatic assembly of DNA molecules up to several hundred kilobases. Nat Methods. 2009 May;6 (5): 343-5). Small-scale isolation of plasmid DNA was done using QIAprep miniprep kit (Qiagen) from 2 mL overnight bacterial culture supplemented with appropriate antibiotics. Large-scale isolation of plasmid DNA was done using the QIAprep maxiprep kit (Qiagen) from 150 mL of overnight bacterial culture supplemented with appropriate antibiotics. All plasmid sequences were confirmed by Sanger sequencing with appropriate sequencing primers (GATC Biotech).

### Chromophore Synthesis

The synthesis of HMBR and HBR-2,5DM were previously reported (Plamont et al., Small fluorescence-activating and absorption-shifting tag for tunable protein imaging in vivo. Proc Natl Acad Sci USA. 2016 Jan. 19;113 (3): 497-502) and Li et al., Dynamic multicolor protein labeling in living cells. Chem Sci. 2017 Aug. 1;8 (8): 5598-5605).

### Cell Culture

HeLa cells (ATCC CRM-CCL2) were cultured in Minimal Essential Media (MEM) supplemented with phenol red, Glutamax I, 1 mM of sodium pyruvate, 1% (vol/vol) of non-essential amino-acids and 10% (vol/vol) fetal calf serum (FCS), at 37° C. in a 5% CO₂ atmosphere. HEK 293T cells (ATCC CRL-3216) were cultured in Dulbecco's Modified Eagle Medium (DMEM) supplemented with phenol red and 10% (vol/vol) FCS at 37° C. in a 5% CO₂ atmosphere. U2OS cells (ATCC HTB-96) were cultured in Dulbecco's modified Eagle's medium (DMEM) supplemented with phenol red and 10% (vol/vol) FCS and 1% (vol/vol) penicillin-streptomycin at 37° C. in a 5% CO₂ atmosphere. For imaging, cells were seeded in uDish IBIDI (Biovalley) coated with poly-L-lysine. Cells were transiently transfected using Genejuice (Merck) according to the manufacturer's protocols for 24 hours prior to imaging. Cells were washed with DPBS (Dulbecco's Phosphate-Buffered Saline), and treated with DMEM medium (without serum and phenol red) supplemented with the compounds (e.g., molecular inducer of proximity) at the indicated concentration.

### Flow Cytometry Analysis

Flow cytometry in HEK 293T cells was performed on a MACSQuant® analyzer equipped with 405 nm, 488 nm and 635 nm lasers and seven channels. To prepare samples, cells were first grown in cell culture flasks, then transiently co-transfected 24 hours after seeding using Genejuice (Merck) according to the manufacturer's protocol for 24 hours. After 24 hours, cells were centrifuged in PBS-BSA (Phosphate-Buffered Saline with 1 mg/mL bovine serum albumin) and resuspended in PBS-BSA supplemented with the appropriate amounts of compounds (e.g., molecular inducer of proximity). For each experiment, 20,000 cells positively expressing mTurquoise2 (ex 434 nm/em 473 nm) and iRFP670 (ex 638 nm/em 660±10 nm) were analyzed with the following parameters: excitation (or ex) 488 nm, emission (or em) 525±20 nm. mTurquoise2 and iRFP670 are transfection markers used to select cells expressing the constructs of interest. Data were analyzed using FlowJo v10.7.1.

### Fluorescence Microscopy

The confocal micrographs were acquired on a Zeiss LSM 980 Laser Scanning Microscope equipped with a plan apochromat 63x/1.4 NA oil immersion objective. ZEN software was used to collect the data. The images were analyzed using Icy (2.4.0.0) and Fiji (Image J). In short, to track fluorescence signal in a specific organelle, a ROI (region of interest) was determined by masking the signal in the ECFP (Enhanced Cyan Fluorescent Protein) channel with the plug-in HK-means with the following parameters: intensity class equals 100, minimum object size (pixels or px) equals 500, maximum object size (px) equals 1500-3000. The signal intensity of the ROI was tracked over time for each channel by using plug-in Active Contours. The background signal was subtracted. Data were processed using GraphPad Prism v9.3.0.

### Results

### Proximity Inducing System

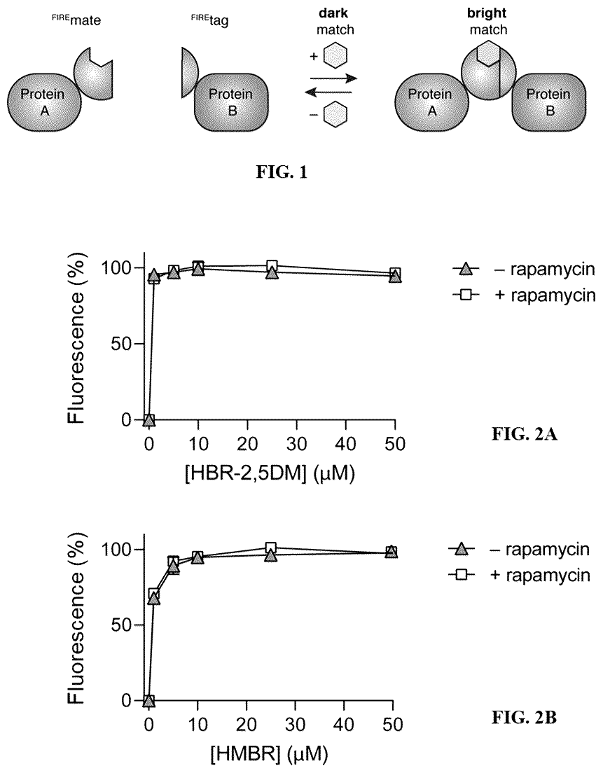

Bisection of a mutated photoactive yellow protein (PYP) gave two fragments: a first polypeptide of 114 amino acid residues having a sequence as set forth in SEQ ID NO: 1 (also referred to as ^{FIRE}mate), and a second polypeptide of 11 amino acid residues having a sequence as set forth in SEQ ID NO: 2 (also referred to as ^{FIRE}tag). As shown on FIG. 1, the two fragments, i.e., the two polypeptides of SEQ ID NO: 1 (i.e., ^{FIRE}mate) and SEQ ID NO: 2 (i.e., ^{FIRE}tag), were surprisingly found to be able to complement and form a fluorescent ternary assembly (also referred to as “FIRE”) with a fluorogen such as HBR-2,5DM or HMBR (also referred to as “the match”). Of note, fluorogens such as HBR-2,5DM or HMBR only emit substantial fluorescence when in an environment constraining their conformation and excluding deexcitation of their excited state, such as the assembly formed with the two polypeptides of SEQ ID NO: 1 (i.e., ^{FIRE}mate) and SEQ ID NO: 2 (i.e., ^{FIRE}tag). Strikingly, the ternary assembly between the first polypeptide of SEQ ID NO: 1 (i.e., ^{FIRE}mate), the second polypeptide of SEQ ID NO: 2 (i.e., ^{FIRE}tag), and the fluorogen was found to occur upon addition of the fluorogen, regardless of any initial proximity between the two polypeptides of SEQ ID NO: 1 and SEQ ID NO: 2. With ^{FIRE}mate and ^{FIRE}tag, FIRE fluorogens such as HBR-2,5DM or HMBR thus act as molecular inducers of proximity.

The formation of a fluorescent ternary assembly was assessed by flow cytometry in HEK293 cells expressing ^{FIRE}mate and ^{FIRE}tag fused to the C-termini of FKBP (FK506-binding protein) and FRB (FKBP-rapamycin-binding domain of mammalian target of rapamycin), respectively. FKBP and FRB are able to dimerize upon addition of rapamycin. Treatment of the HEK293 cells with rapamycin is thus expected to induce the dimerization of FKBP and FRB, and subsequently to induce proximity between ^{FIRE}tag and ^{FIRE}mate. As shown on FIG. 2, treatment of the HEK293 cells with the fluorogens HBR-2,5DM (FIG. 2A) or HMBR (FIG. 2B) induced the formation of a ternary fluorescent assembly at all tested fluorogen concentrations (1, 5, 10, 25 or 50 μM as indicated). Of note, upon addition of the fluorogen, the formation of a ternary fluorescent assembly occurred regardless of the presence or absence of rapamycin, showing that initial proximity between ^{FIRE}tag and ^{FIRE}mate was not necessary to induce the formation of the ternary fluorescent assembly between ^{FIRE}tag, ^{FIRE}mate, and HBR-2,5DM or HMBR. Accordingly, even in the absence of rapamycin, ^{FIRE}tag, ^{FIRE}mate, and HBR-2,5DM or HMBR were able to induce the dimerization of FKBP and FRB, that is to say to induce proximity between FKBP and FRB.

Proximity does not Occur in the Absence of Fluorogens

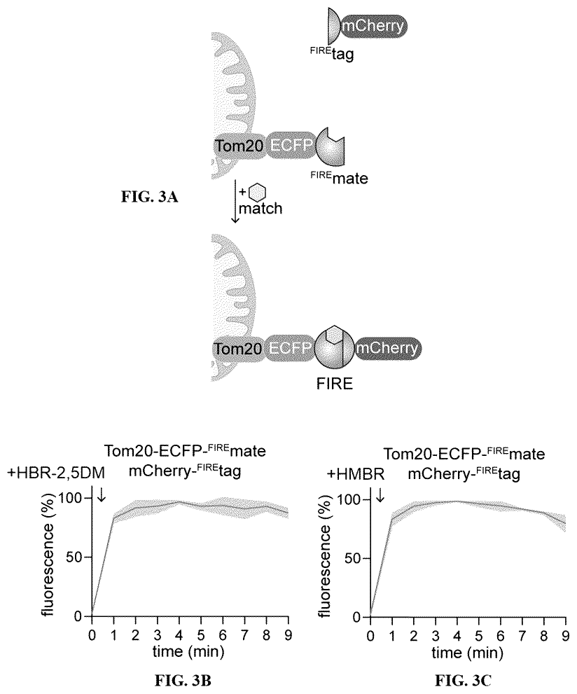

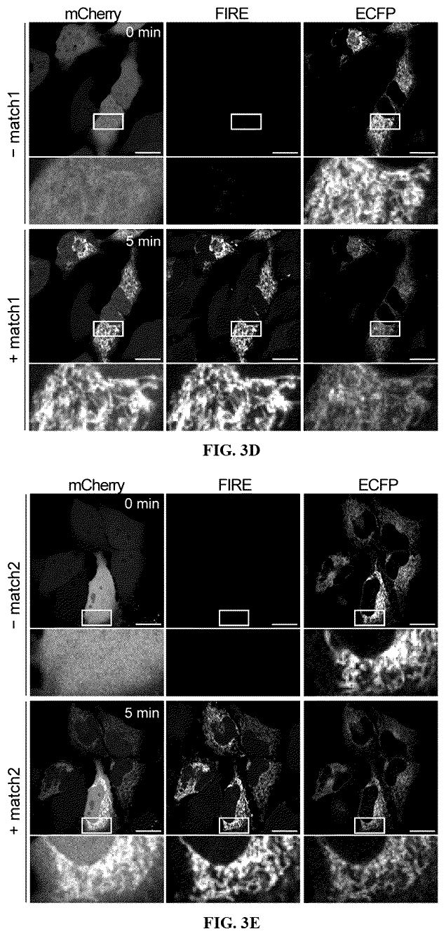

To test whether or not ^{FIRE}mate and ^{FIRE}tag self-complement in absence of fluorogens, the two fragments were spatially separated to analyze their mutual affinity. As shown on FIG. 3A, ^{FIRE}tag was fused to the C-terminus of the red fluorescent protein mCherry for diffuse cytosolic expression, and ^{FIRE}mate was fused to the C-terminus of the N-terminal domain of the mitochondrial outer membrane protein TOM20 (TOM20_1-34), so that pFAST1-114 faced the cytosol. An enhanced cyan fluorescent protein (ECFP) was inserted between ^{FIRE}mate and TOM20_1-34 to monitor the localization of the resulting fusion protein (blue fluorescence). Fluorescence imaging of HeLa cells co-expressing the two fusion proteins in absence of fluorogen showed a diffused cytosolic localization of the mCherry-^{FIRE}tag fusion protein (red fluorescence) and a mitochondria localization of the TOM20-ECFP-^{FIRE}mate fusion protein (blue fluorescence), suggesting that ^{FIRE}mate and FIRE ^{FIRE}tag do not spontaneously complement (FIGS. 3B-E).

Addition of HBR-2,5DM triggered a very rapid translocation of the mCherry-^{FIRE}tag fusion protein from the cytosol to mitochondria, as observed by the cytosol-to-mitochondria shift of the red fluorescence (i.e., mCherry fluorescence), and led to the simultaneous appearance of a strong green fluorescent signal (i.e., FIRE signal corresponding to the HBR-2,5DM fluorescence)) at the mitochondria (FIGS. 3B, D). These observations suggest that fluorogen-induced complementation of ^{FIRE}mate and ^{FIRE}tag is able to mediate the interaction of mCherry and TOM20-ECFP in a sustainable manner, and to simultaneously image the formed complex. Similar results were observed with the fluorogen HMBR (FIGS. 3C, E).

These experiments thus demonstrate that fluorogens such as HBR-2,5DM or HMBR can induce the rapid and sustainable proximity/interaction of two proteins fused to ^{FIRE}mate and ^{FIRE}tag, respectively, and light up the recruitment process through formation of a sustainable ternary fluorescent assembly between ^{FIRE}mate, ^{FIRE}tag, and a fluorogen such as HBR-2,5DM or HMBR.

### Induced Proximity is Extremely Rapid and Stable

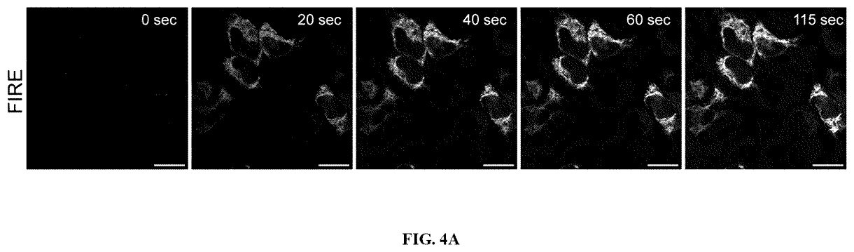

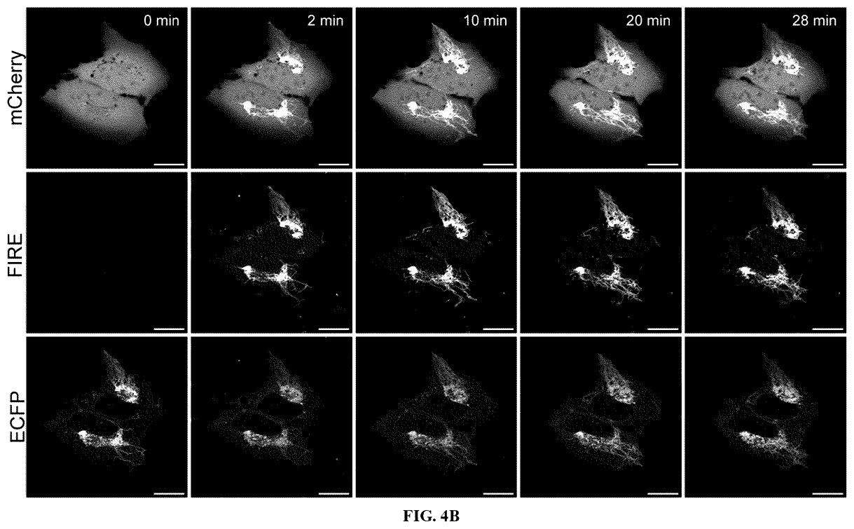

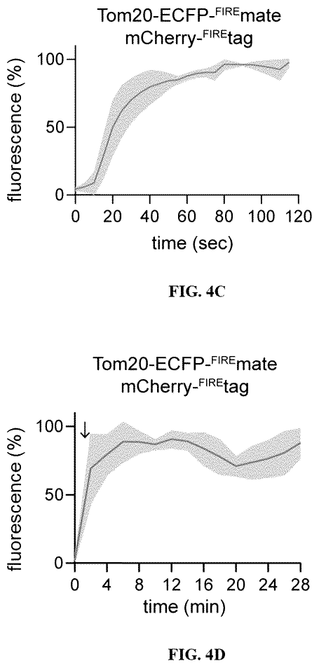

Additional time-lapse confocal microscopy experiments were conducted in HeLa cells co-expressing the two fusion proteins (i.e., mCherry-^{FIRE}tag and TOM20-ECFP-^{FIRE}mate) and treated with HBR-2,5DM. As shown on FIGS. 4A, C, these experiments indicated that the formation of the ternary fluorescent assembly occurred with a half-time of about 20 seconds. This suggests that the process is extremely rapid and almost only limited by the cellular uptake and diffusion of the fluorogen. Furthermore, the time-lapse confocal microscopy experiments also indicated that the ternary fluorescent assembly was stable on a long period of time as observed on FIGS. 4B, D, with HBR-2,5DM fluorescence detected for more than 25 minutes.

Induced Proximity does not Depend on the Positioning within a Fusion Protein

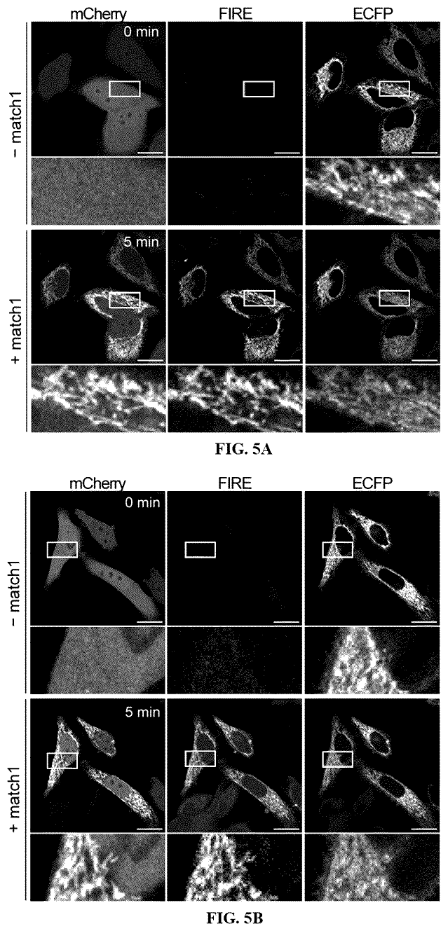

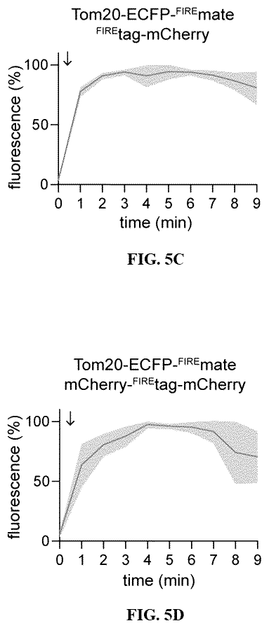

To examine the impact of the ^{FIRE}tag positioning on the efficiency of formation of the fluorescent ternary assembly, the ^{FIRE}tag was fused at the N-terminus of mCherry (^{FIRE}tag-mCherry), or inserted in between two mCherry (mCherry-^{FIRE}tag-mCherry). The TOM20-ECFP-^{FIRE}mate fusion protein as described above was co-expressed in HeLa cells with either the ^{FIRE}tag-mCherry fusion protein or with the mCherry-^{FIRE}tag-mCherry fusion protein. Addition of HBR-2,5DM resulted in a similar rapid and stable translocation of both the ^{FIRE}tag-mCherry fusion protein (FIGS. 5A, C) and the mCherry-^{FIRE}tag-mCherry fusion protein (FIGS. 5B, D) to mitochondria, demonstrating that the positioning of the ^{FIRE}tag into a fusion protein has no impact on the efficiency of formation of the fluorescent ternary assembly.

Use of the Proximity Inducing System to Control Localization of a Protein within a Cell

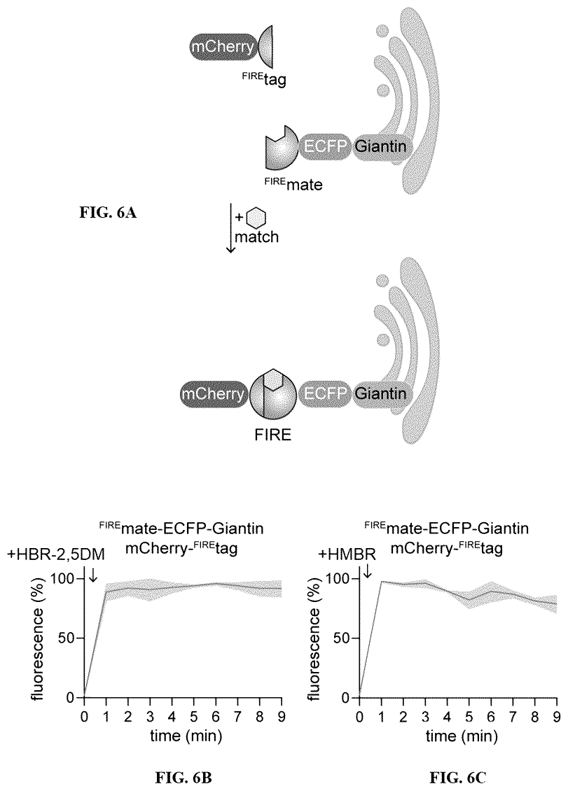

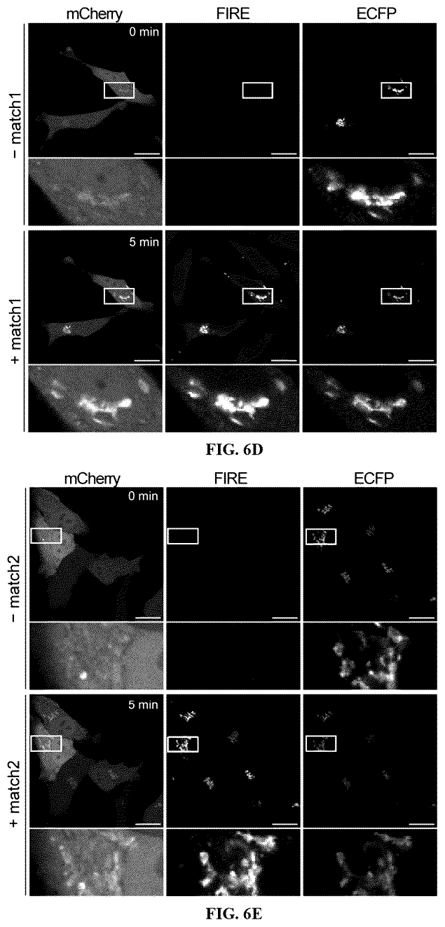

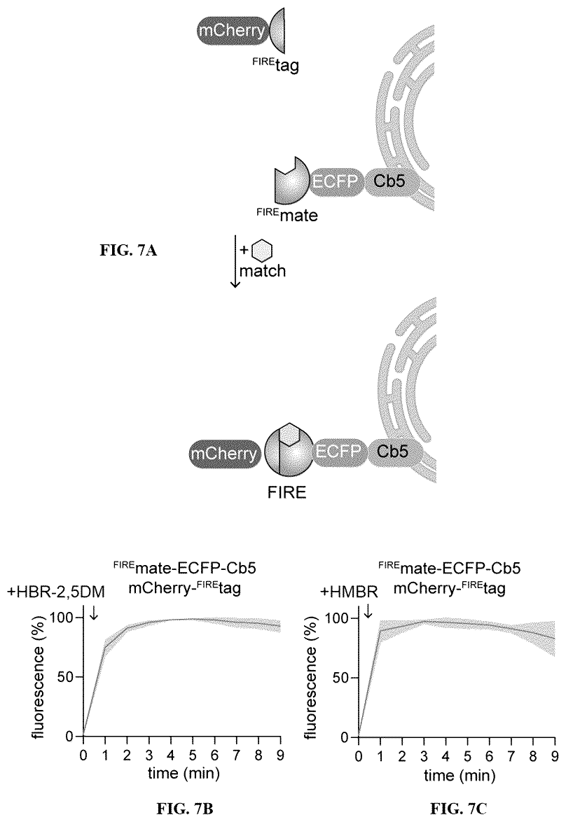

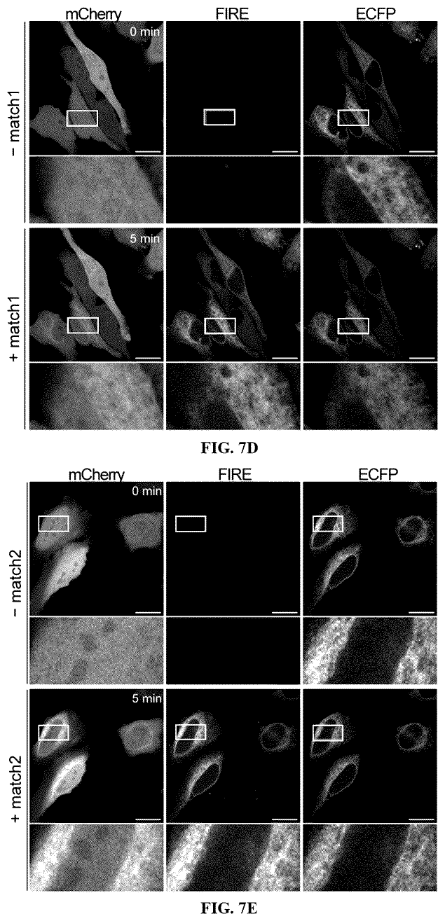

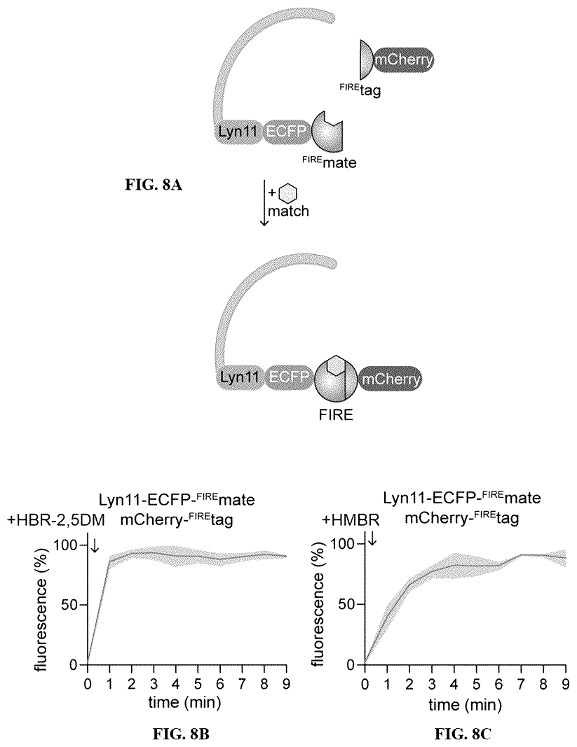

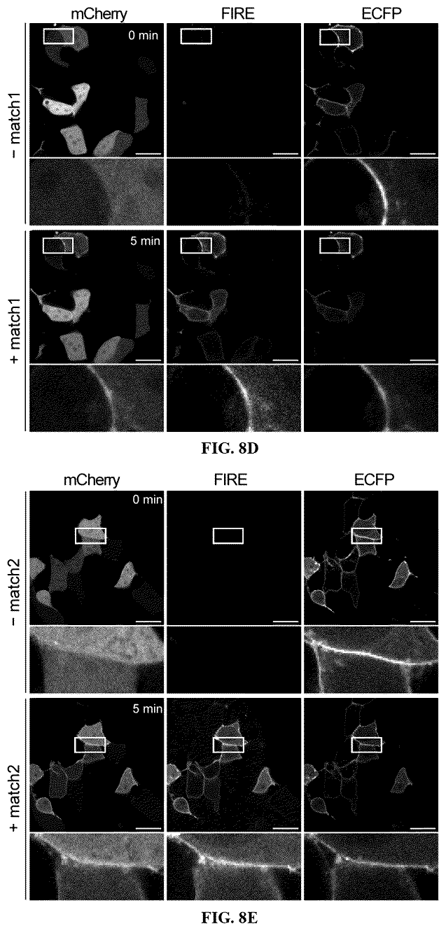

To further evaluate the scope of the proximity inducing system described herein and show its versatility, ^{FIRE}mate was anchored at the membrane of various organelles or cellular structures so that ^{FIRE}mate faced the cytosol. ^{FIRE}mate was thus fused:
to the N-terminus of the transmembrane domain (amino acid residues at positions 3131-3259) of Giantin for location at the cis-medial rims of the Golgi apparatus (FIG. 6A),
to the N-terminus of the transmembrane domain (amino acid residues at positions 100-134) of the cytochrome b5 (Cb5) for location at the endoplasmic reticulum membrane (FIG. 7A), and
to the C-terminus of the Lyn11 inner membrane targeting sequence for location at the plasma membrane (FIG. 8A).

For each construct, an ECFP was inserted between ^{FIRE}mate and the other protein domain/sequence to monitor the localization of the resulting fusion protein (blue fluorescence). Each fusion protein was co-expressed in mammalian cells (HeLa cells or HEK293 cells as indicated) together with mCherry-^{FIRE}tag (red fluorescence). Addition of either HBR-2,5DM (FIGS. 6B&D, 7B&D, 8B&D) or HMBR (FIGS. 6C&E, 7C&E, 8C&E) resulted in rapid translocation of mCherry-^{FIRE}tag to the organelle where the ^{FIRE}mate fusion protein was expressed, as observed by the cytosol-to-organelle shift of the red fluorescence and the simultaneous appearance of green fluorescence (i.e., FIRE signal corresponding to the HBR-2,5DM or HMBR fluorescence) at the organelle localization, demonstrating efficient formation of a fluorescent ternary assembly between ^{FIRE}tag, ^{FIRE}mate, and either HBR-2,5DM or HMBR. The translocation of mCherry-^{FIRE}tag occurred with very similar kinetics regardless of the organelle or cellular structure (i.e., Golgi apparatus, endoplasmic reticulum membrane, or plasma membrane).

### Induced Proximity is Reversible

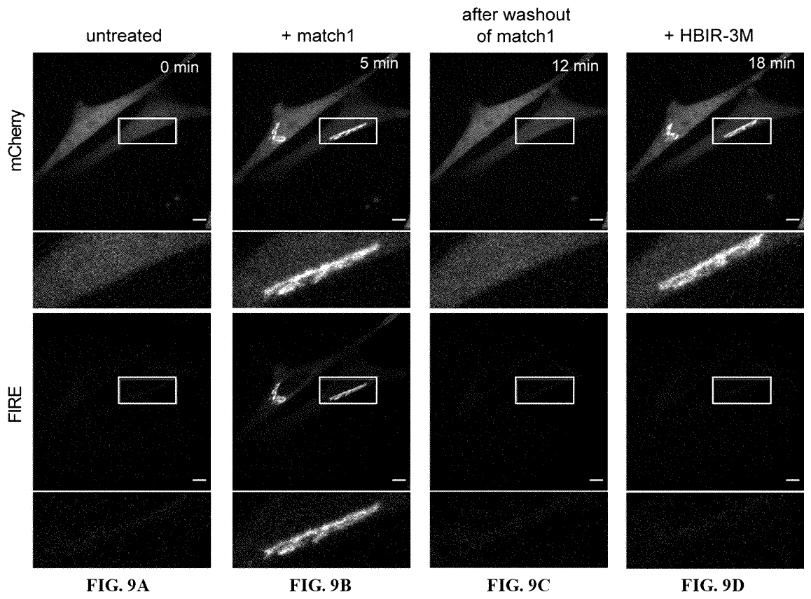

To test whether formation of the fluorescent ternary assembly could be reversed though removal of the fluorogen, the fusion proteins mCherry-^{FIRE}tag (red fluorescence) and ^{FIRE}mate-ECFP-Giantin (blue fluorescence) were co-expressed in Hela cells (FIG. 9A). Formation of a fluorescent ternary assembly between ^{FIRE}tag, ^{FIRE}mate and HBR-2,5DM was induced by addition of 10 μM of HBR-2,5DM (FIG. 9B). The washing of cells with HBR-2,5DM-free medium led to rapid dissociation of the fluorescent ternary assembly and simultaneous release of mCherry into the cytosol (FIG. 9C), demonstrating the full reversibility of the fluorescence ternary assembly. The ability to reverse the fluorescence ternary assembly at will allowed to induce repeated catch and release cycles through sequential addition and removal of HBR-2,5DM. The ability to control protein proximity through addition and removal of a molecular inducer of proximity is unprecedented and opens great prospect to control the proximity of two proteins with high temporal resolution.

Proximity can be Induced with a Non-Fluorogenic Chromophore

Next, experiments were carried out to investigate whether other chromophores could act as molecular inducer of proximity and provide new properties. In particular, non-fluorogenic chromophores, such as HBIR-3M, were investigated. HBIR-3M is able to form very tight non-fluorescent ‘invisible’ assembly with ^{FIRE}tag and ^{FIRE}mate. The fusion proteins mCherry-^{FIRE}tag (red fluorescence) and ^{FIRE}mate-ECFP-Giantin (blue fluorescence) were co-expressed in HeLa cells as described above. Through cycles of sequential addition and removal, HBIR-3M was shown to induce the proximity of the proteins fused to ^{FIRE}mate (i.e., ECFP-Giantin) and ^{FIRE}tag (i.e., mCherry) as efficiently as HBR-2,5DM (FIG. 9D).

### Caged Proximity Inducing System

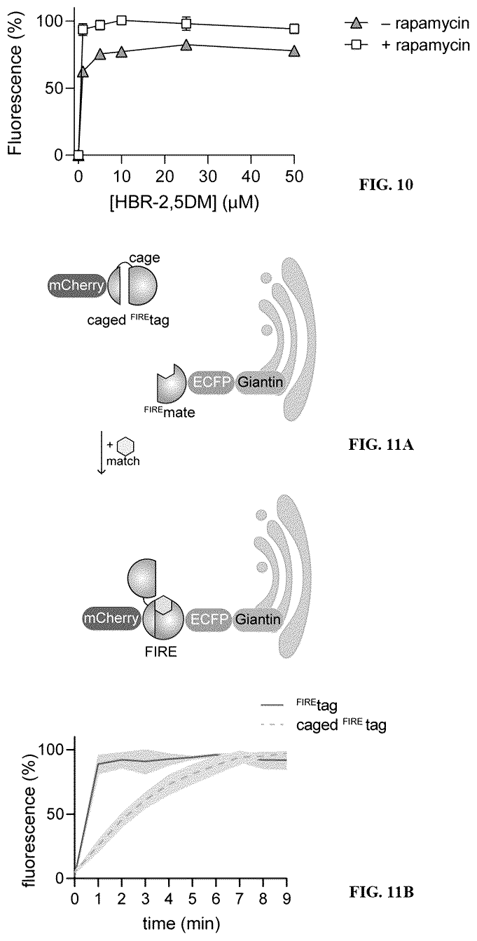

Although, as indicated above, no self-complementation between ^{FIRE}tag and ^{FIRE}mate could be observed in the absence of fluorogen (see FIG. 3), a caged FIREt was designed to prevent any interaction with ^{FIRE}mate in the absence of fluorogen, even at high expression levels. To cage ^{FIRE}tag, the N-terminal domain 2-114 of Halorhodospira halophila photoactive yellow protein (PYP) C69G (corresponding to SEQ ID NO: 21) was fused to ^{FIRE}tag. The intramolecular interaction between ^{FIRE}tag and the N-terminal domain 2-114 of H. halophila PYP C69G induces the folding of the caged^{FIRE}tag protein, thus preventing any intermolecular interaction of the caged^{FIRE}tag with ^{FIRE}mate. The fusion proteins FKBP-caged^{FIRE}tag and FRB-^{FIRE}mate were co-expressed in HEK293T cells. The cells were then treated with the fluorogens HBR-2,5DM at various concentrations (1, 5, 10, 25 or 50 μM as indicated), in the presence or absence of rapamycin. As shown on FIG. 10, analysis by flow cytometry showed that caged^{FIRE}tag could still form a fluorescent ternary assembly with ^{FIRE}mate and HBR-2,5DM, even in absence of rapamycin, that is to say regardless of any initial proximity between caged^{FIRE}tag and ^{FIRE}mate. Of note, in accordance with the caging of ^{FIRE}tag, higher concentrations of HBR-2,5DM were required to reach full ternary assembly in absence of rapamycin. These results suggest that the formation of a highly stable ternary assembly provides the driving force required to uncage ^{FIRE}tag for efficient complementation with ^{FIRE}mate via a mutually exclusive folding mechanism.

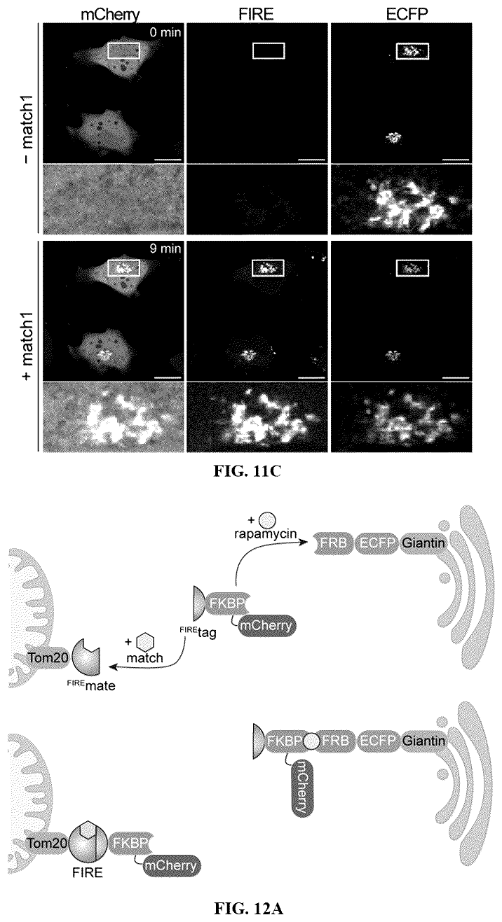

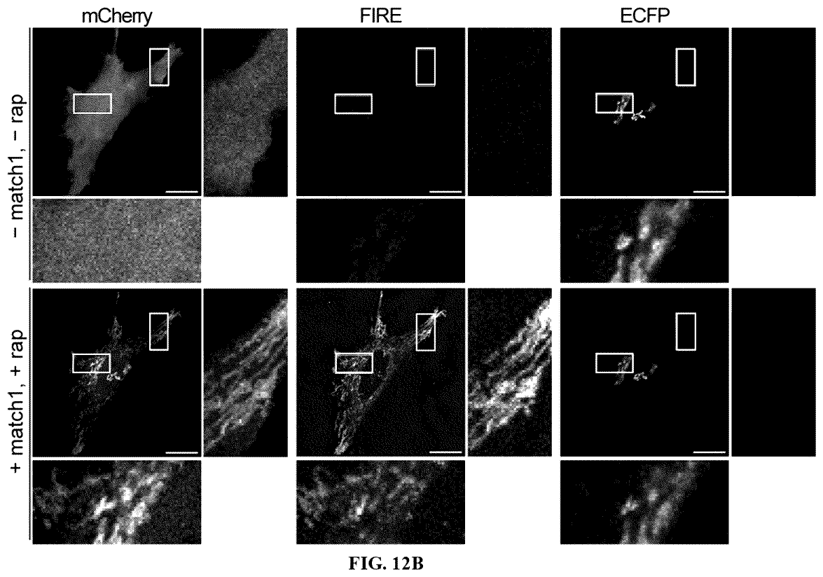

To further verify that caged^{FIRE}tag and ^{FIRE}mate do not interact together, the fusion proteins mCherry-caged^{FIRE}tag and ^{FIRE}mate-ECFP-Giantin were co-expressed in HeLa cells (FIG. 11A). The cells were then imaged in absence or in presence of HBR-2,5DM (FIG. 11C). In absence of HBR-2,5DM, the two fusion proteins did not colocalize, confirming that caged^{FIRE}tag and ^{FIRE}mate exhibit no significant binding affinity. However, treatment with HBR-2,5DM resulted in efficient recruitment of mCherry-caged^{FIRE}tag to the Golgi, demonstrating efficient complementation of caged^{FIRE}tag and ^{FIRE}mate. Of note, the need for unfolding caged^{FIRE}tag for efficient complementation led however to slower kinetics, as compared to fluorescent ternary assembly between ^{FIRE}tag, ^{FIRE}mate, and HBR-2,5DM (FIG. 11B).

The Proximity Inducing System can be Used Simultaneously with a CIP System

In order to control more than one protein-protein interaction, the combination of the “FIRE” proximity inducing system described herein with the FRB-FKBP-rapamycin CIP system was next investigated (FIG. 12). The chimeric fusion protein mCherry-FKBP-^{FIRE}tag was expressed together with both the FRB-EGFP-Giantin and the TOM20-^{FIRE}mate fusion proteins in HeLa cells (FIG. 12A). Simultaneous addition of HBR-2,5DM and rapamycin triggered the translocation of mCherry-FKBP-^{FIRE}tag to the Golgi apparatus and to the mitochondria (FIG. 12B), demonstrating that the two systems are orthogonal and can be used for controlling two interactions simultaneously.

### Use of the Proximity Inducing System to Control Nucleocytoplasmic Trafficking of a Protein

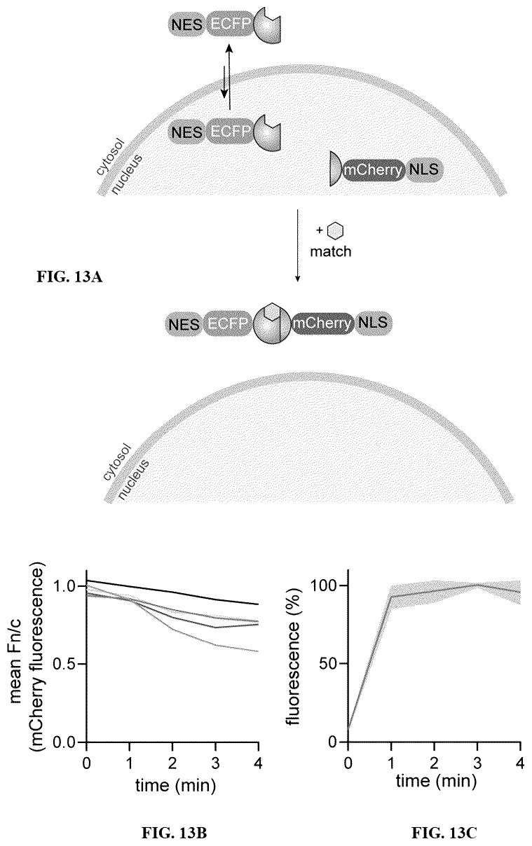

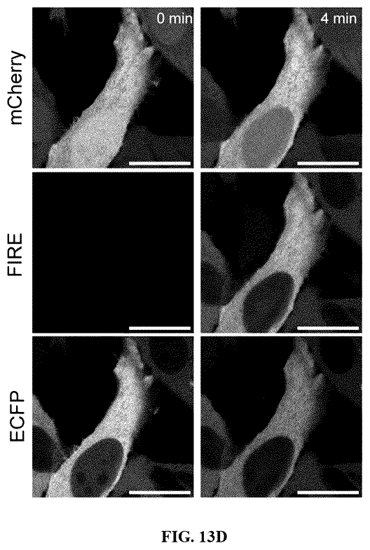

To further demonstrate the potential of the proximity inducing system described herein, an assay was developed to control nucleocytoplasmic trafficking of proteins (FIG. 13). Nucleocytoplasmic trafficking is tightly regulated through mechanisms involving nuclear localization signals (NLS) and nuclear export signal (NES). Control of nuclear protein export was first investigated using NLS-mCherry-^{FIRE}mate as an NLS-containing cargo molecule and NES-ECFP-^{FIRE}tag as the export NES-containing cargo partner (FIG. 13A). In absence of HBR-2,5DM (FIG. 13D-0 min), NLS-mCherry-^{FIRE}mate was almost equally distributed between the nucleus and the cytosol. The export cargo molecule NES-ECFP-^{FIRE}tag could freely enter the nuclear compartment by diffusion. As shown on FIG. 13D (4 min), addition of HBR-2,5DM resulted in efficient cytoplasmic localization of the formed protein complex, as judged by a nuclear-to-cytoplasmic shift of the NLS-mCherry-^{FIRE}mate fluorescence (FIG. 13B) and the appearance of a strong cytoplasmic green fluorescence (FIG. 13C). These results confirm that in protein assemblies containing both NLS and NES, the export activity prevails over the import activity (Busch et al. Quantification of nuclear protein transport using induced heterodimerization. Traffic. 2009 September;10 (9): 1221-7).

1-15. (canceled)

16. A method for inducing proximity between two biological molecules in a sample or an assay relying on the induction of proximity between two biological molecules in a sample, said method or assay comprising the following steps:
obtaining a first biological molecule coupled to a first polypeptide, wherein said first polypeptide comprises an amino acid sequence as set forth in SEQ ID NO: 1 or an amino acid sequence having at least about 70% identity with SEQ ID NO: 1, or a truncated fragment thereof comprising at least 89 consecutive amino acid residues from the C-terminal end of SEQ ID NO: 1 or of the sequence having at least about 70% identity with SEQ ID NO: 1;
obtaining a second biological molecule coupled to a second polypeptide, wherein said second polypeptide comprises an amino acid sequence as set forth in SEQ ID NO: 2 or an amino acid sequence having at least about 70% identity with SEQ ID NO: 2, or a truncated fragment thereof comprising at least 8 consecutive amino acid residues from the N-terminal end of SEQ ID NO: 2 or of the sequence having at least about 70% identity with SEQ ID NO: 2;
adding a molecular inducer of proximity to the sample;
wherein, upon addition of the molecular inducer of proximity, the first polypeptide, the second polypeptide and the molecular inducer of proximity form a ternary assembly, with the concentration of molecular inducer of proximity required to reach half maximal assembly in the sample being of less than about 5 μM,
thereby inducing proximity between the first and the second biological molecules present in the sample.

17. The method or assay according to claim 16, wherein the molecular inducer of proximity is fluorogenic and wherein the method or assay further comprises detecting a fluorescence resulting from the formation of the ternary assembly between the first polypeptide, the second polypeptide and the fluorogenic molecular inducer of proximity.

18. The method or assay according to claim 16, wherein the first polypeptide consists of an amino acid sequence as set forth in SEQ ID NO: 1, and the second polypeptide consists of an amino acid sequence as set forth in SEQ ID NO: 2.

19. The method or assay according to claim 16, wherein the molecular inducer of proximity is a compound of formula (I) 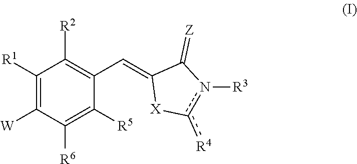
or a salt and/or solvate thereof;
wherein
R¹, R², R⁵ and R⁶ each independently represents H, halo, hydroxyl, aryl, alkyl, cycloalkyl, heteroalkyl or heterocycloalkyl,
wherein said aryl, alkyl, cycloalkyl, heteroalkyl or heterocycloalkyl is optionally substituted by at least one group selected from halo, hydroxyl, oxo, nitro, amido, carboxy, amino, cyano, haloalkoxy and haloalkyl;
R³ represents a non-binding doublet, H, halo, hydroxyl, aryl, alkyl, cycloalkyl, heteroalkyl or heterocycloalkyl,
wherein said aryl, alkyl, cycloalkyl, heteroalkyl or heterocycloalkyl is optionally substituted by at least one group selected from halo, hydroxyl, oxo, nitro, amido, carboxy, amino, cyano, haloalkoxy and haloalkyl;
R⁴ represents a single or a double bound, interrupted or terminated by one S, O or N heteroatom, wherein said heteroatom is optionally substituted by at least one group selected from H, hydroxyl, aryl, alkyl, cycloalkyl, heteroalkyl and heterocycloalkyl,
wherein said aryl, alkyl, cycloalkyl, heteroalkyl or heterocycloalkyl is optionally substituted by at least one group selected from halo, hydroxyl, oxo, nitro, amido, carboxy, amino, cyano, haloalkoxy and haloalkyl;
W represents OH, SH, NHR⁷ or NR⁷R⁸, wherein R⁷ and R⁸ each independently represents H, halo, hydroxyl, aryl, alkyl, cycloalkyl, heteroalkyl or heterocycloalkyl,
wherein said aryl, alkyl, cycloalkyl, heteroalkyl or heterocycloalkyl is optionally substituted by at least one group selected from halo, hydroxyl, oxo, nitro, amido, carboxy, amino, cyano, haloalkoxy and haloalkyl;
X represents O, S or NH; and
Z represents O, S or NH.

20. The method or assay according to claim 16, wherein said assay is for controlling the localization, the transport and/or the function of a biological molecule.

21. The method or assay according to claim 16, wherein said assay is for inducing the degradation of a biological molecule.

22. The method or assay according to claim 16, wherein said assay is for detecting the co-occurrence of two biological molecules in the sample.

23. The method or assay according to claim 16, wherein the first and second biological molecules are proteins.

24. A proximity inducing system comprising two polypeptides and a molecular inducer of proximity, wherein:
a first polypeptide comprises an amino acid sequence as set forth in SEQ ID NO: 1 or an amino acid sequence having at least about 70% identity with SEQ ID NO: 1, or a truncated fragment thereof comprising at least 89 consecutive amino acid residues from the C-terminal end of SEQ ID NO: 1 or of the sequence having at least about 70% identity with SEQ ID NO: 1; and
a second polypeptide comprises an amino acid sequence as set forth in SEQ ID NO: 2 or an amino acid sequence having at least about 70% identity with SEQ ID NO: 2, or a truncated fragment thereof comprising at least 8 consecutive amino acid residues from the N-terminal end of SEQ ID NO: 2 or of the sequence having at least about 70% identity with SEQ ID NO: 2,
wherein the first polypeptide, the second polypeptide and the molecular inducer of proximity form a ternary assembly, with the concentration of molecular inducer of proximity required to reach half maximal assembly being of less than about 5 μM.

25. The proximity inducing system according to claim 24, wherein the molecular inducer of proximity is fluorogenic.

26. The proximity inducing system according to claim 24, wherein the molecular inducer of proximity is a compound of formula (I) 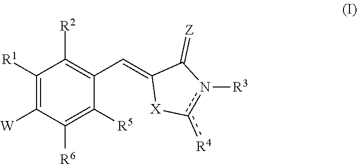
or a salt and/or solvate thereof;
wherein
R¹, R², R⁵ and R⁶ each independently represents H, halo, hydroxyl, aryl, alkyl, cycloalkyl, heteroalkyl or heterocycloalkyl,
wherein said aryl, alkyl, cycloalkyl, heteroalkyl or heterocycloalkyl is optionally substituted by at least one group selected from halo, hydroxyl, oxo, nitro, amido, carboxy, amino, cyano, haloalkoxy and haloalkyl;
R³ represents a non-binding doublet, H, halo, hydroxyl, aryl, alkyl, cycloalkyl, heteroalkyl or heterocycloalkyl,
wherein said aryl, alkyl, cycloalkyl, heteroalkyl or heterocycloalkyl is optionally substituted by at least one group selected from halo, hydroxyl, oxo, nitro, amido, carboxy, amino, cyano, haloalkoxy and haloalkyl;
R⁴ represents a single or a double bound, interrupted or terminated by one S, O or N heteroatom, wherein said heteroatom is optionally substituted by at least one group selected from H, hydroxyl, aryl, alkyl, cycloalkyl, heteroalkyl and heterocycloalkyl,
wherein said aryl, alkyl, cycloalkyl, heteroalkyl or heterocycloalkyl is optionally substituted by at least one group selected from halo, hydroxyl, oxo, nitro, amido, carboxy, amino, cyano, haloalkoxy and haloalkyl;
W represents OH, SH, NHR⁷ or NR⁷R⁸, wherein R⁷ and R⁸ each independently represents H, halo, hydroxyl, aryl, alkyl, cycloalkyl, heteroalkyl or heterocycloalkyl,
wherein said aryl, alkyl, cycloalkyl, heteroalkyl or heterocycloalkyl is optionally substituted by at least one group selected from halo, hydroxyl, oxo, nitro, amido, carboxy, amino, cyano, haloalkoxy and haloalkyl;
X represents O, S or NH; and
Z represents O, S or NH.

27. The proximity inducing system according to claim 24, wherein the first polypeptide consists of an amino acid sequence as set forth in SEQ ID NO: 1, and the second polypeptide consists of an amino acid sequence as set forth in SEQ ID NO: 2.

28. A cell or cell line comprising two biological molecules, wherein:
a first biological molecule is coupled to a first polypeptide, wherein said first polypeptide comprises an amino acid sequence as set forth in SEQ ID NO: 1 or an amino acid sequence having at least about 70% identity with SEQ ID NO: 1, or a truncated fragment thereof comprising at least 89 consecutive amino acid residues from the C-terminal end of SEQ ID NO: 1 or of the sequence having at least about 70% identity with SEQ ID NO: 1;
a second biological molecule is coupled to a second polypeptide, wherein said second polypeptide comprises an amino acid sequence as set forth in SEQ ID NO: 2 or an amino acid sequence having at least about 70% identity with SEQ ID NO: 2, or a truncated fragment thereof comprising at least 8 consecutive amino acid residues from the N-terminal end of SEQ ID NO: 2 or of the sequence having at least about 70% identity with SEQ ID NO: 2,
wherein in presence of a molecular inducer of proximity, the first polypeptide, the second polypeptide and the molecular inducer of proximity are able to form a ternary assembly, with the concentration of molecular inducer of proximity required to reach half maximal assembly being of less than about 5 μM.

29. The cell or cell line according to claim 28, wherein the first polypeptide consists of an amino acid sequence as set forth in SEQ ID NO: 1, and the second polypeptide consists of an amino acid sequence as set forth in SEQ ID NO: 2.

30. The cell or cell line according to claim 28, wherein the first and second biological molecule are proteins.


SEQUENCE LISTING (23 sequences)

SEQ ID NO: 1 - protein, 114 aa, synthetic construct
MEHVAFGSEDIENTLANMDDEQLDRLAFGVIQLDGDGNILLYNAAEGDITGRDPKQVIGKNFFKDVAPGTDTPEFYGKFKEGAASGNLNTMFEWTIPTSRGPTKVKVHLKKALS

SEQ ID NO: 2 - protein, 11 aa, synthetic construct
GDRYWVFVKRV

SEQ ID NO: 3 - protein, 114 aa, synthetic construct
METVRFGGDDIENSLAKMDDKKLDELAFGAIQLDANGKIIQYNAAEGGITGRDPKSVIGKNFFTEVAPCTQSKEFQGRFKEGVSSGELNTMFEYVFDYQMTPTKVKVHMKKAIS

SEQ ID NO: 4 - protein, 114 aa, synthetic construct
METVRFGGDDIENALANMDDKKLDTLAFGAIQLDANGKIIQYNAAEGGITGRDPKSVIGKNFFTDVAPCTQSKEFQGRFKEGVKNGDLNTMFEYVFDYQMTPTKVKVHMKKALS

SEQ ID NO: 5 - protein, 114 aa, synthetic construct
METVRFGGDDIENSLAKMDDKALDKLAFGAIQLDGNGKIIHYNAAEGTITGRDPKTVIGKNFFTDVAPCTQSKEFQGRFKEGVQKGDLNTMFEYVFDYQMKPTKVKVHMKKAMT

SEQ ID NO: 6 - protein, 114 aa, synthetic construct
MEIVQFGSDDIENTLSKMSDDKLNDIAFGAIQLDASGKIIQYNAAEGDITGRDPGAVVGKNFFNEVAPCTNSPEFKGRFDEGVKNGNLNTMFEYVFDYEMQPTKVKVHMKKALT

SEQ ID NO: 7 - protein, 114 aa, synthetic construct
MELLSFGADNIENSLAKMSKGDLNKLAFGAIQLNAQGKILQYNAAEGDITGRKPTEVIGKNFFLEVAPCTNRTEFKGRFDQGIKSGNLNTMFEYTFDYEMKPTKVKVHMKKALV

SEQ ID NO: 8 - protein, 114 aa, synthetic construct
MEMIKFGQDDIENAMADMGDAQIDDLAFGAIQLDETGTILAYNAAEGELTGRSPQDVIGKNFFKDIAPCTDTEEFGGRFREGVANGDLNAMFEYVFDYQMQPTKVKVHMKRAIT

SEQ ID NO: 9 - protein, 114 aa, synthetic construct
METVRFGGDDIENSLAKMDDKKLDELAFGAIQLDANGKIIQYNAAEGGITGRDPKSVIGKNFFTEVAPGTQSKEFQGRFKEGVSSGELNTMFEWMIPTSRGPTKVKVHMKKAIS

SEQ ID NO: 10 - protein, 114 aa, synthetic construct
METVRFGGDDIENALANMDDKKLDTLAFGAIQLDANGKIIQYNAAEGGITGRDPKSVIGKNFFTDVAPGTQSKEFQGRFKEGVKNGDLNTMFEWMIPTSRGPTKVKVHMKKALS

SEQ ID NO: 11 - protein, 114 aa, synthetic construct
METVRFGGDDIENSLAKMDDKALDKLAFGAIQLDGNGKIIHYNAAEGTITGRDPKTVIGKNFFTDVAPGTQSKEFQGRFKEGVQKGDLNTMFEWMIPTSRGPTKVKVHMKKAMT

SEQ ID NO: 12 - protein, 114 aa, synthetic construct
MEIVQFGSDDIENTLSKMSDDKLNDIAFGAIQLDASGKIIQYNAAEGDITGRDPGAVVGKNFFNEVAPGTNSPEFKGRFDEGVKNGNLNTMFEWMIPTSRGPTKVKVHMKKALT

SEQ ID NO: 13 - protein, 114 aa, synthetic construct
MELLSFGADNIENSLAKMSKGDLNKLAFGAIQLNAQGKILQYNAAEGDITGRKPTEVIGKNFFLEVAPGTNRTEFKGRFDQGIKSGNLNTMFEWMIPTSRGPTKVKVHMKKALV

SEQ ID NO: 14 - protein, 114 aa, synthetic construct
MEMIKFGQDDIENAMADMGDAQIDDLAFGAIQLDETGTILAYNAAEGELTGRSPQDVIGKNFFKDIAPGTDTEEFGGRFREGVANGDLNAMFEWMIPTSRGPTKVKVHMKRAIT

SEQ ID NO: 15 - protein, 11 aa, synthetic construct
GDTYWIFVKRL

SEQ ID NO: 16 - protein, 11 aa, synthetic construct
GDTFWIFVKRL

SEQ ID NO: 17 - protein, 11 aa, synthetic construct
GDSFWIFVKRL

SEQ ID NO: 18 - protein, 11 aa, synthetic construct
GDTYWVFVKRL

SEQ ID NO: 19 - protein, 11 aa, synthetic construct
DDTYWVFVKRV

SEQ ID NO: 20 - protein, 11 aa, synthetic construct
GDSYWIFVKRV

SEQ ID NO: 21 - protein, 113 aa, synthetic construct
EHVAFGSEDIENTLAKMDDGQLDGLAFGAIQLDGDGNILQYNAAEGDITGRDPKQVIGKNFFKDVAPGTDSPEFYGKFKEGVASGNLNTMFEYTFDYQMTPTKVKVHMKKALS

SEQ ID NO: 22 - DNA, 342 bp, synthetic construct
  source 1..342: mol_type = other DNA
atggagcatgttgcctttggcagtgaggacatcgagaacactctggccaatatggacgacgaacaactggataggttggcctttggcgtaattcagctcgatggtgacgggaatatcctgctgtacaatgctgctgaaggggacatcactggcagagatcccaaacaggtgattgggaagaacttcttcaaggatgttgcacctggaacggatactcccgagttttacggcaaattcaaggaaggcgcagcgtcagggaatctgaacaccatgttcgaatggacgataccgacaagcaggggaccaaccaaggtcaaggtgcacttgaagaaagccctttcc

SEQ ID NO: 23 - DNA, 33 bp, synthetic construct
  source 1..33: mol_type = other DNA
ggtgacagatattgggtctttgtgaaacgggtg